(19) United States
(12) Patent Application Publication
(10) Pub. No.: US 2025/0295872 A1
(43) Pub. Date: Sep. 25, 2025

(54) METHOD OF MANUFACTURE OF ARTICLE FOR DRY POWDER INHALER WITH UPSTREAM RESILIENT ELEMENT

(71) Applicant: PHILIP MORRIS PRODUCTS S.A., Neuchâtel (CH)

(72) Inventor: Antonio CONSOLANTE, Casalecchio di Reno (IT)

(21) Appl. No.: 18/869,033

(22) PCT Filed: May 26, 2023
(86) PCT No.: PCT/IB2023/055428

§ 371 Date: Nov. 25, 2024

(30) Foreign Application Priority Data
May 27, 2022 (EP) 22175773.5

(51) Int. Cl. A61M 15/06; A61M 15/00

(52) U.S. Cl. CPC A61M 15/06; A61M 15/0021; A61M 15/003; A61M 2202/064; A61M 2207/00

(57) ABSTRACT

The present disclosure provides a method for manufacturing an inhaler article having an upstream resilient element (101) for insertion into a holder to form an inhaler system for providing a dry powder to the lungs of a user, the manufacturing method comprising: moving a ribbon (402) of resilient material from a feeder reel (401) to a cutting stage (405); presenting the ribbon of resilient material to the cutting stage; cutting at least one disk of resilient material from the ribbon of resilient material to form at least one cut disk of resilient material; presenting the at least one cut disk of resilient material to a glue stage (501); dispensing at least one glue ring (502); applying the at least one glue ring to the at least one cut disk of resilient material; and, presenting the at least one cut disk of resilient material having an applied glue ring to an upstream end (120) of an inhaler article; affixing the at least one cut disk of resilient material having an applied glue ring to the upstream end of an inhaler article to form an inhaler article having an affixed upstream resilient element.

The present disclosure relates to a method for manufacturing an inhaler article for insertion into a holder to form an inhaler system for delivering an active ingredient in the form of a dry powder to the lungs of a user. Such inhaler systems may have two parts, an inhaler article and a holder. The inhaler article may contain a capsule filled with dry powder. When combined, the inhaler article and the holder form an inhaler system.

Dry powder inhaler articles are not always fully suitable for providing airflow which is optimized for dry powder drug delivery during use, while also minimizing leakage of dry powder or loss of capsules from the inhaler articles before and after use.

It may be desirable to provide a dry powder inhaler article which is closed before and after use and is also capable of opening during use. It would be desirable to provide a dry powder inhaler article which is sufficiently closed before and after use to prevent loss of capsules contained in the dry powder inhaler article. It would be desirable to provide a dry powder inhaler article which is sufficiently closed before and after use to prevent loss of dry powder contained in a capsule contained in the dry powder inhaler article. It would be desirable to provide a dry powder inhaler article which is sufficiently open during use to allow optimal airflow through the dry powder inhaler article during use. It would be desirable to provide a dry powder inhaler article having an upstream resilient element that is closed before and after use and is open during use. It may be desirable to provide a dry powder inhaler article having a resilient element at the upstream end to contain a capsule or dry powder.

It may be desirable to provide dry powder inhaler articles containing a capsule that are protected from opening to reveal or release the capsule. For example, it may be desirable to provide a dry powder inhaler article containing a capsule where the capsule is not visible. If the capsule is visible inside the inhaler article, it may be tempting to open the inhaler article to release the capsule.

It may be desirable to provide an inhaler article which protects against removing the capsule from the inhaler article. The capsule may be released from the inhaler article intentionally or accidentally. It may be desirable to provide a dry powder inhaler article having an element at the upstream end to prevent intentional or unintentional release of the capsule from the inhaler article.

Appropriate manufacturing methods are desirable to ensure that the resilient element is affixed to the inhaler article so that the capsule is safely contained inside the inhaler article.

It may be desirable to provide a dry powder inhaler article with improved looks at the upstream end of the inhaler article. For example, adding an element to the upstream end of the inhaler article may cover the upstream end of the inhaler article. The added element may cover inconsistencies that may occur at the upstream end of the inhaler article and improve the way that the upstream end of the inhaler article looks. The upstream element may provide a pleasing end face. In addition, the upstream element may provide indicia. Indicia may indicate origin, flavor, strength or other information to the user. Indicia may be color, symbols, or a combination of color and symbols.

Manufacturing methods are desirable to ensure that the resilient element is affixed to the inhaler article so that the inhaler article has a pleasing end face.

The inhaler article may be a tubular article having an upstream end and a downstream end. The upstream end may be inserted into the holder. The downstream end, the mouth end, may be engaged by the mouth of a user to enable inhalation of the dry powder into the lungs of a user. The inhaler article contains a dose, or multiple doses, of dry powder active ingredient. The dry powder may be contained in a capsule inside the inhaler article. The inhaler article may be a disposable article. To use the inhaler system, a user may insert an inhaler article into the holder, activate the system to release dry powder from the capsule, inhale dry powder, and then remove the spent inhaler article from the holder. Activating the system may include piercing the capsule.

It may be desirable that the inhaler article is structured and arranged to optimize dry powder delivery from the inhaler system to the lungs of a user during use. In addition, before and after use, it may be desirable to ensure that the capsule, and the dry powder active ingredient, are safely contained in the inhaler article. That is, it may be desirable that the capsule does not fall out of the inhaler article, before or after use. It is also desirable that the dry powder contained in the capsule does not fall out of the inhaler article before or after use.

An inhaler article having a resilient element at the upstream end of the inhaler article, where the resilient element is in a closed position before insertion into the holder, where the resilient element opens to an open position in response to an opening force applied to the resilient element when the inhaler article is inserted into the holder in use, and where the resilient element closes to a closed position upon removal of the inhaler article from the holder after use is provided. In an aspect, the upstream end of the capsule cavity of the inhalation article may be a resilient element forming an openable closed end of the inhaler article.

Traditional smoking articles do not have an element on the upstream end of the smoking article. The upstream end of a traditional smoking article is the lit end.

The upstream end in traditionally smoking article is set afire, and aerosol released from the firing of aerosol-generating substrates are inhaled. Any element at the upstream end of a traditional smoking article would be set afire, and aerosol released from burning such an element at the upstream end would be inhaled.

In the dry powder inhaler article disclosed herein, the upstream end is not lit in use. Therefore, the upstream end of the inhaler article can take on utility that is not desired in traditional smoking devices.

The present disclosure provides a method of manufacturing an inhaler article having a resilient element at the upstream end of the inhaler article. The method of manufacture includes the steps of moving a ribbon of resilient material from a feeder reel to a cutting stage, presenting the ribbon of resilient material to the cutting stage; cutting at least one disk of resilient material from the ribbon of resilient material; presenting the at least one cut disk to a glue stage; dispensing at least one glue ring; applying the at least one glue ring to the at least one disk of resilient material; presenting the at least one cut disk having an applied glue ring to an upstream end of an inhaler article; affixing the at least one disk having an applied glue ring to the upstream end of the inhaler article to form an inhaler article having an upstream resilient element. The upstream resilient element is affixed to the upstream end of the inhaler article.

The present disclosure provides a method of manufacture wherein the inhaler article comprises a tubular body extending along a longitudinal axis from a downstream mouthpiece end to an upstream end; a capsule cavity containing a capsule; wherein an upstream boundary of the capsule cavity is defined by the resilient element and wherein the affixed resilient element retains he capsule in the capsule cavity. In embodiments, the downstream mouthpiece end comprises a blocker element. The blocker element may be a filter. The tubular body may be made of cardboard.

The resilient element of the present disclosure has the advantage that it ensures that the capsule in the inhaler article is contained in the inhaler article before use. The resilient element opens when inserted into the holder to provide an airflow path from the upstream end of the inhaler article to the downstream mouthpiece end of the inhaler article. As air moves through the inhaler article, dry powder released from the capsule is entrained in the airflow and is delivered to the mouthpiece end of the inhaler article to be inhaled by the user. That is, the resilient element is structured and arranged to open to optimize dry powder delivery from the inhaler system to the lungs of a user during use. After use, upon removal of the inhaler article from the holder, the resilient element closes to retain the capsule inside the inhaler article. It is advantageous for the inhaler article to open when inserted into the holder during use to provide sufficient airflow to deliver the pharmaceutically active agent to the lungs of a user. It is advantageous for the inhaler article to be closed before use to prevent the capsule from falling out of the inhaler article before use. It is advantageous for the inhaler article to be closed after use to prevent the capsule from falling out of the inhaler article after use. It is advantageous for the inhaler article to be sufficiently closed after use, after the inhaler article has been activated to prevent the capsule from falling out of the inhaler article after use. The resilient element allows for both opening and closing of the upstream end of the inhaler, both before and after use of the inhaler article. The resilient element can open to provide sufficient airflow, when engaged with the holder, to optimize dry powder delivery to the lungs of a user and can close to prevent the capsule from falling out of the inhaler before and after insertion of the inhaler article into the holder. The resilient element on the upstream end of the inhaler article provides both the advantage of enabling sufficient airflow and also protecting from unwanted loss of pharmaceutically active agents from the inhaler article before and after use.

According to an aspect, the resilient element of the inhaler article is a resilient element that is pre-cut to form flaps. The flaps of the resilient element fold when inserted into the holder to provide an opening which forms an airflow path from the upstream end of the inhaler article to the downstream mouthpiece end of the inhaler article. As air moves through the inhaler article, dry powder released from the capsule is entrained in the airflow and is delivered to the mouthpiece end of the inhaler article to be inhaled by the user. That is, the flaps of the resilient element open to optimize dry powder delivery from the inhaler system to the lungs of a user during use. After use, upon removal of the inhaler article from the holder, the resilient element closes to retain the capsule inside the inhaler article. The resilient element may be pre-cut to form at least 4 flaps. The resilient element may be pre-cut to form at least 6 flaps. The cuts may extend between about 65% to about 95% of the diameter of the resilient element. Resilient material, provided at the upstream end of the inhaler article in the form of flaps, may open and close in a manner suitable to provide an open inhaler article during use when inserted into a holder and be closed before and after use to prevent the capsule from falling out of the inhaler article.

The open inhaler article provides an enlarged airflow path to improve airflow through the inhaler system, and to provide a higher dose of powder to the user. If the upstream end of the inhaler article is closed prior to use, the piercing pin introduces a hole in the upstream end of the inhaler article. The hole made by the piercing pin in the upstream end of an inhaler article is generally related to the size of the piercing pin. In general, a hole made by a piercing pin is not large enough to enable sufficient airflow through the inhaler system to provide an appropriate dose of powder to the user. That is, a small hole in the upstream end of the inhaler article in an inhaler system introduces significant resistance to draw (RTD) because of a narrow aperture related to the size of the piercing pin. This would lead to an inhaler system that does not have sufficient airflow to provide an appropriate dose of powder released from a capsule to a user. One solution to this RTD challenge is to provide an inhaler article having an upstream end that is open. Providing an inhaler article having an upstream end with flaps or an aperture that open in response to an opening force provided by the holder allows for a larger opening and creates an airflow path that is not limited by a pin-hole-sized region in the airflow path.

The present disclosure provides an inhaler article for insertion into a holder to form an inhaler system for providing a dry powder to the lungs of a user. The inhaler article may be a tubular body extending along a longitudinal axis from a downstream mouthpiece end to an upstream end; a capsule cavity within the tubular body between the downstream mouthpiece end and the upstream end; the capsule cavity containing a capsule. The upstream of the inhaler article may have a resilient element. When inhaler article is inserted into the holder, the resilient element opens to an open position in response to an opening force provided by the holder. Upon removal of the inhaler article from the holder, the opening force is removed from the inhaler article. Upon removal of the opening force from the inhaler article, the resilient element closes to a closed position, sufficiently closed to retain the capsule in the capsule cavity of the inhaler article between the resilient element at the upstream end and the downstream mouthpiece end. The resilient element is made from elastic material suitable for opening and closing as described.

According to an aspect of the present disclosure, the inhaler article may be a tubular body extending along a longitudinal axis from a downstream mouthpiece end to an upstream end, having a capsule cavity within the tubular body between the downstream mouthpiece end and the upstream end, the capsule cavity containing a capsule. In an aspect, the downstream mouthpiece end has a blocker element. In an aspect the blocker is a filter element. The blocker element functions to ensure that the capsule does not exit the tubular body from the downstream end, before, during and after use. When the blocker is a filter, the filter element ensures that the pharmaceutically active powder delivered to the lungs of the user is powder that is small enough to pass through the filter. This ensures that the pharmaceutically active powder is suitable for delivery to the lungs of the user.

According to an aspect, the resilient element is a disk. In an aspect, the resilient element is round. In an aspect, the resilient element is cut from a ribbon of resilient material to form a disk. Resilient element may be cut from a ribbon of resilient by a stamp cutter. Resilient elements may be cut from a ribbon of resilient material one at a time. That is, the cutter that cuts disks of resilient material from the ribbon of resilient material may have one cutting head. Or, resilient elements may be cut from a ribbon of resilient material two at a time. That is, the cutter that cuts disks of resilient material from the ribbon of resilient material may have two cutting heads. Or, resilient elements may be cut from a ribbon of resilient material three at a time. That is, the cutter that cuts disks of resilient material from the ribbon of resilient material may have three cutting heads. Or, resilient elements may be cut from a ribbon of resilient material more than three at a time. That is, the cutter that cuts disks of resilient material from the ribbon of resilient material may have more than three cutting heads.

In an aspect, the resilient element is an applied or affixed resilient element. The resilient element may be affixed to upstream end of the inhaler article. The resilient element may be affixed to the upstream end of the inhaler article by any process suitable to affix a resilient element to an inhaler article including gluing, heat sealing, pressing, friction fitting, or other means. The resilient element may be affixed to the upstream end of the inhaler article by gluing. The resilient element may be affixed to the upstream end of the inhaler article by heat sealing. The resilient element may be affixed to the upstream end of the inhaler article by pressing the resilient element into the inhaler article. The resilient element may be affixed to the upstream end of the inhaler article by friction fit. The resilient element may be affixed to the upstream end of the inhaler article by any suitable means.

In an aspect, before the resilient element is applied or affixed to the upstream end of the inhaler article, a capsule is inserted into the inhaler article. That is, the resilient element is affixed to the upstream end of the inhaler article which contains a capsule.

In an aspect, the resilient element is affixed to the upstream end of the inhaler article by gluing. In an aspect, a glue stage may dispense a ring of glue. The ring of glue may be slightly smaller than the circumference of the disk of resilient material. After the disk of resilient material is cut, the disk of resilient material may be presented to the glue stage having a dispensed ring of glue. By pressing the cut disk of resilient material against the dispensed ring of glue on the glue stage, the ring of glue can be applied to the cut disk of resilient material. Cut disks may be transported to a gluing station by a holder. Glue may be introduced to a gluing stage. For example, glue may be applied to the stage. Glue may be pressure-fed to glue rings, sized slightly smaller than the diameter of resilient element disks at the glue stage. In embodiments, glue may be presented to the glue ring by pressing the glue into the glue ring from a reservoir of glue below the glue stage. Alternatively, glue may be presented in dots, a discontinuous ring, a thick ring, a thin ring or any other shape to provide glue to the glue station. Appropriate glues may include starch adhesives such as dextrin, casein-based adhesive, polyamide blue, hot melt glue, cyanoacrylate, organic glues, or any other suitable glue.

According to an aspect, the resilient element of the inhaler article is a resilient element that is cut to form flaps. The cuts in the disk of resilient material may be provided by the same cutter that cuts the disk of resilient material. In an aspect, the cuts are made by a stamp cutter. These cuts do not extend all the way to the external circumference of the disk of resilient material. That is, each cut in the disk of resilient material crosses through the center of the disk of resilient material but does the cut all the way through the disk of resilient material. When more than one cut is made through the center of the disk of resilient material, a disk having sectors is formed. Sectors are the shape enclosed between an arc and the two radii at either end of that arc. Because the sectors are cut from resilient material, these sectors form flaps. Flaps are sectors which can move.

The flaps of the resilient element fold when inserted into the holder to provide an opening which forms an airflow path from the upstream end of the inhaler article to the downstream mouthpiece end of the inhaler article. As air moves through the inhaler article, dry powder released from the capsule is entrained in the airflow and is delivered to the mouthpiece end of the inhaler article to be inhaled by the user. That is, the flaps of the resilient element open to optimize dry powder delivery from the inhaler system to the lungs of a user during use. After use, upon removal of the inhaler article from the holder, the resilient element closes to retain the capsule inside the inhaler article.

The resilient element may be cut with at least 2 cuts to form at least 4 flaps. The resilient element may be cut with at least 3 cuts to form at least 6 flaps. The resilient element may be cut with at least 4 cuts to form at least 8 flaps. The cuts may extend between about 65% to about 95% of the diameter of the resilient element. Resilient material, provided at the upstream end of the inhaler article in the form of flaps, may open and close in a manner suitable to provide an open inhaler article during use when inserted into a holder and be closed before and after use to prevent the capsule from falling out of the inhaler article.

In an aspect, the flaps may open to an open position by folding the cut flaps into the capsule cavity of the inhaler article in response to an opening force. The opening force may be provided by the holder. That is, a portion of the holder may fit inside the upstream end of the inhaler article when the inhaler article is inserted into the holder. As the portion of the holder fits inside the inhaler article, the holder may force the flaps to fold into the capsule cavity. When the flaps are folded into the capsule cavity of the inhaler article, an aperture is opened, and an airflow path is created. The inhaler article of any of claims 6-9 wherein the resilient element having cuts forming flaps opens to an open position by folding the cut flaps into the capsule cavity in response to an opening force. Inserting the inhaler article into the holder may provide an opening force which forces the flaps of the cut resilient element into the capsule cavity of the inhaler article.

In an aspect, the resilient element may be cut to provide a central aperture in the disk of resilient material. The cuts in the disk of resilient material may be provided by the same cutter that cuts the disk of resilient material. In an aspect, the cuts are made by a stamp cutter. In an aspect, the central aperture has a diameter which comprises less than 30% of a diameter of the resilient element.

The cutter may provide cutting of both cuts (which form flaps) and a central aperture.

In an aspect, the resilient element of the inhaler article is a resilient element that has a central aperture which opens to an open position by stretching open in response to an opening force. Stated another way, in an aspect, the resilient element of the inhaler article is an annular resilient element of resilient material which opens to an open position by stretching open in response to an opening force. The central aperture diameter may be less than 30% of the resilient element diameter. The opening force may be provided by the holder. The opening force may be provided by the movement of prongs of the holder into the central aperture of the upstream end of the inhaler article when the inhaler article is inserted into the holder. Inserting the inhaler article into the holder may provide an opening force which forces the central aperture to stretch open in response to the opening force.

After the resilient element has been cut from the ribbon of resilient material, the ribbon of resilient material may proceed away from the cutting stage to be collected by a take-up reel.

The resilient element of the inhaler article may be made of resilient material. The resilient element of the inhaler article may be made of, for example, silicon, latex, plastic, paper, paper tape, laminated layered PLA on a paper layer, or cardboard. The resilient element may be made of silicon, latex, rubber or a combination. The resilient element of the inhaler article may be made of, for example, silicon. The resilient element of the inhaler article may be made of, for example, latex. The resilient element of the inhaler article may be made of, for example, plastic. The resilient element of the inhaler article may be made of, for example, paper. The resilient element of the inhaler article may be made of, for example, aluminum foil. The resilient element of the inhaler article may be made of, for example, paper tape. The resilient element of the inhaler article may be made of, for example, laminated layered PLA on a paper layer. The resilient element of the inhaler article may be made of, for example, cardboard. The resilient element of the inhaler article may be made of, for example, rubber. The resilient element of the inhaler article may be made of, for example, a combination of materials. The resilient element of the inhaler article may be made of, for example, any suitable resilient material.

In an aspect, when the resilient element is in the open position, engaged in the holder, the resulting opening or aperture provides unrestricted, or less restricted airflow into the capsule cavity. In an aspect, the holder is configured to provide swirling or rotational inhalation airflow to the inhaler article. In an aspect, the holder comprises a housing defining a housing cavity, a moveable cap configured to retain the inhaler article within the housing cavity, the moveable cap is movable within the housing cavity along the longitudinal axis of the housing, wherein the moveable cap comprises prongs and a piercing end. Prongs of the moveable cap extend into the resilient element at the upstream end of the inhaler article when the inhaler article is engaged in the holder. A piercing element, affixed to the inner surface of the bottom of the holder extends through the piercing end of the moveable cap. When the moveable cap moves in relation to the piercing element, the inhaler article is moved so that it is presented to the piercing element. The piercing element then pierces the capsule in the inhaler article to release pharmaceutically active dry powder from the capsule.

In an aspect, the capsule contains pharmaceutically active dry powder. The pharmaceutically active dry powder may be nicotine.

Typically, the capsule would not contain filler material of the tobacco industry. Typically, filler material of the tobacco industry is tobacco, for example chopped or shredded small pieces of tobacco plant leaves or stems. It would be undesirable to provide small pieces of tobacco leaves or stems directly to the lungs of a user. Providing small pieces of tobacco leaves or stems to the lungs of the user would not provide pharmaceutically active nicotine to the user. Providing small pieces of plant material to the lungs of a user would likely be harmful to the user.

In an aspect, the resilient element may comprise indicia. In embodiments, the indicia may be color. In embodiments the indicia may be one or more symbols. In embodiments, the ribbon of resilient material may be colored. In embodiments, the ribbon of resilient material may comprise one or more symbols. Or, the ribbon of resilient material may comprise both color and one or more symbols. When the ribbon of resilient material comprises color, the cut disk of resilient material, when applied or affixed to the upstream end of an inhaler article is colored. When the ribbon of resilient material comprises one or more symbols, the cut disk of resilient material, when applied or affixed to the upstream end of an inhaler article has one or more symbols. Color and symbols are indicia. Indicia may indicate origin of the goods, flavor, strength, or other information.

As used herein, the singular forms “a”, “an”, and “the” also encompass embodiments having plural referents, unless the content clearly dictates otherwise.

As used herein, “or” is generally employed in its sense including “and/or” unless the content clearly dictates otherwise. The term “and/or” means one or all of the listed elements or a combination of any two or more of the listed elements.

As used herein, “have”, “having”, “include”, “including”, “comprise”, “comprising” or the like are used in their open-ended sense, and generally mean “including, but not limited to”. It will be understood that “consisting essentially of”, “consisting of”, and the like are subsumed in “comprising,” and the like.

The words “preferred” and “preferably” refer to embodiments of the invention that may afford certain benefits, under certain circumstances. However, other embodiments may also be preferred, under the same or other circumstances. Furthermore, the recitation of one or more preferred embodiments does not imply that other embodiments are not useful and is not intended to exclude other embodiments from the scope of the disclosure, including the claims.

As used herein, “providing”, in the context of providing an apparatus or system, means manufacturing the apparatus or system, purchasing the apparatus or system, or otherwise obtaining the apparatus or system.

As used herein, “or” is generally employed in its sense including “and/or” unless the content clearly dictates otherwise. The term “and/or” means one or all of the listed elements or a combination of any two or more of the listed elements.

Any direction referred to herein such as “top”, “bottom”, “left”, “right”, upper”, “lower”, and other directions or orientations are described herein for clarity and brevity but are not intended to be limiting of an actual device or system. Devices and systems described herein may be used in a number of directions and orientations.

As used herein, “downstream” and “proximal” mean the mouthpiece end of the inhaler article. “Downstream” and “proximal” mean the end of the tubular inhaler article intended to be contacted by the mouth of a user. “Upstream” and “distal” mean the opposite end of the inhaler article. “Upstream” and “distal” mean the end of the tubular inhaler article intended to be inserted into the holder.

The term “nicotine” refers to nicotine and nicotine derivatives such as free-base nicotine, nicotine salts and the like.

As used herein the term “closed” means sufficiently closed to retain a capsule inside the inhaler article before and after use. “Closed” also means sufficiently closed to reduce loss of pharmaceutically active powder from the inhaler article before or after use. The inhaler article may be less closed after use than before use, but may still be considered closed if the function of reducing loss of the contents of the inhaler article is provided.

As used herein the term “use” means the steps of inserting an inhaler article into a holder, initiating an airflow through the holder and the inhaler article, and inhaling pharmaceutically active powder. “Use” may optionally include the additional step of removing the inhaler article from the holder.

Inhaler systems are used to provide pharmaceutically active dry powder to the lungs of a user. The present disclosure provides a system and in particular an inhaler article, structured and arranged to contain active dry powder before and after use, and allow dry powder to leave, or be delivered from the inhaler article during use. The inhaler article has two ends, an upstream end and a downstream end. The downstream end is the mouthpiece end. During use, the user puts the downstream end of the inhaler article in the user's mouth and inhales.

The upstream end of the inhaler article is inserted into a holder during use. When the upstream end of the inhaler article is inserted into the holder, the upstream end of the inhaler article opens. When the upstream end of the inhaler article is inserted into the holder, the inhaler article plus the holder form an inhaler system. The inhaler article is inserted into the holder during use. The upstream end of the inhaler article opens during use to allow air to flow through the inhaler article and release pharmaceutically active dry powder to the user. The upstream end of the inhaler article opens when the inhaler article is inserted into the holder. The holder provides an opening force to the inhaler article to open the inhaler article to release pharmaceutically active powder from the inhaler article during use.

To prevent the pharmaceutically active dry powder from falling out of the inhaler article before use, the upstream end of the inhaler article is closed before use. After use, after the inhaler article is removed from the holder, the upstream end of the inhaler article closes to prevent the pharmaceutically active dry powder from falling out of the inhaler after use. The upstream end of the inhaler article is open when it is inserted into a holder and closed before and after the inhaler article is inserted into the holder.

To enable the upstream end of the inhaler article to be closed before use, open during use, and then closed again after use, the upstream end of the inhaler article is a resilient element. The resilient element is closed before use, open during use, and then comes back to a closed position after use. The resilient element is closed before use, opens when the inhaler article is inserted into the holder, and then closes again when the inhaler article is removed from the holder.

The inhaler article may be any shape or size. For ease of insertion into a holder, the inhaler article may be a tubular article. The inhaler article may be a tubular body extending along a longitudinal axis from a downstream mouthpiece end to an upstream end. The inhaler article may be cylindrical. The inhaler article contains pharmaceutically active powder.

The inhaler article may resemble a smoking article or cigarette in size and shape. The inhaler article may be a tubular body extending along the longitudinal axis of the inhaler article. The inhaler article may have a substantially uniform outer diameter along the length of the elongated body. The inhaler body may have a circular cross-section that may be uniform along the length of the elongated body. The inhaler body may have an outer diameter in a range from about 6 mm to about 10 mm, or from about 7 mm to about 10 mm, or about 7 mm to about 9 mm, or about 7 mm to about 8 mm or about 7.2 mm. The inhaler body may have a length (along the longitudinal axis) in a range from about 40 mm to about 80 mm, or from about 40 mm to about 70 mm, or about 40 mm to about 50 mm, or about 45 mm.

The inhaler article may be a tubular body extending along a longitudinal axis from a downstream mouthpiece end to an upstream end. Between the downstream mouthpiece end and the upstream end is a central cavity.

The downstream end of the inhaler article is the mouthpiece end. That is, the downstream end of the inhaler article is intended to engage with the mouth of a user to allow the contents of the inhaler article to be inhaled by a user. The downstream end is structured and arranged for engagement with the mouth of a user. The downstream end is sized and shaped for engagement with the mouth of a user. The downstream end of the inhaler article acts as a conduit to provide pharmaceutically active powder to the mouth and lungs of a user. The downstream end of the inhaler article has a stiffness or hardness, a size, and a shape for engagement with the mouth of a user. The downstream end of the inhaler article may have a blocker. The blocker may be an element of the inhaler article that provides a stiffness or hardness. The stiffness or hardness of the blocker is appropriate for insertion into the mouth of a user during use of the inhaler system. The blocker may have a size and a shape for engagement with the mouth of a user.

In addition, the blocker may be a filter element. The filter element may have an internal structure that allows smaller particles to flow through the filter element and blocks larger particles from flowing through the filter element. The filter element may act to filter larger particles of pharmaceutically active powder as they flow out of the inhaler article. The filter element may act to control the size of particles of pharmaceutically active powder as they flow out of the inhaler article. The filter element may allow particles of pharmaceutically active powder to flow out of the inhaler article to a user. The filter element may allow particles of pharmaceutically active powder that are small enough to flow through the filter element out of the inhaler article to a user. The filter element may be formed of a cellulose acetate material. The filter element may be cellulose tow. The filter may be formed of a biodegradable material.

The blocker element may extend from the central cavity to the mouthpiece end of the inhaler article. The blocker element may extend from the capsule cavity to the mouthpiece end of the inhaler article. The filter element may have a length in a range from about 10 mm to about 30 mm, preferably from about 15 mm to about 25 mm and more preferably from about 20 mm to about 22 mm.

The inhaler article has an upstream end. The upstream end has a resilient element. The resilient element of the inhaler article may have cuts which allow the resilient element to open when prongs are inserted into the resilient element. These cuts may be referred to as cuts or cuts. These cuts can be described as radial cuts or diametric cuts. Two diametric cuts, results in the formation of four flaps in the resilient element. Two diametric cuts, extending partially across the diameter of the resilient element, is the same as four radial cuts, extending partially across the radius of the resilient element. The cuts may extend in a range of 65% to 95% of the diameter of the resilient element.

When the inhaler article having a resilient element having cuts (and flaps) is inserted into a holder, the prongs of the holder push the flaps into the inside of the inhaler article. When the prongs of the holder push the flaps into the inside of the inhaler article, the inhaler article is open. The prongs of the holder provide an opening force to open the upstream end of the inhaler article. When the inhaler article is open, an airflow passage is provided which improves airflow through the inhaler system. When the inhaler article is withdrawn from the holder, the prongs are withdrawn from the inhaler article and the flaps return to their position at the upstream end of the inhaler article. When the opening force is withdrawn, the inhaler article closes. When the inhaler article is removed from the holder, the opening force is removed from the upstream end of the inhaler article. When the opening force is removed from the inhaler article, the resilient element returns to a closed position. Upon removal of the opening force, the resilient element closes to a closed position. While the resilient element having a cuts and flaps may not be fully closed before or after use, it is partially closed before and after use. It is not open after use.

The resilient element of the inhaler article may have a central aperture which opens to an open position by stretching open in response to an opening force. Stated another way, in an aspect, the resilient element of the inhaler article is an annular resilient element of resilient material which opens to an open position by stretching open in response to an opening force. The central aperture diameter may be less than 30% of the resilient element diameter. The opening force may be provided by the holder. The opening force may be provided by the movement of prongs of the holder into the central aperture of the upstream end of the inhaler article when the inhaler article is inserted into the holder. Inserting the inhaler article into the holder may provide an opening force which forces the central aperture to stretch open in response to the opening force. In addition, the resilient element returns to a closed position when the opening force is removed. When the inhaler article is removed from the holder, the opening force is removed from the upstream end of the inhaler article. When the opening force is removed from the inhaler article, the resilient element returns to a closed position. Upon removal of the opening force, the resilient element closes to a closed position. While the resilient element having a central aperture may not be fully closed before or after use, it is partially closed before and after use. It is not open after use.

The resilient element of the inhaler article may have a combination of cuts and a central aperture which opens to an open position by stretching open in response to an opening force. Stated another way, in an aspect, the resilient element of the inhaler article is an annular resilient element of resilient material which opens to an open position by stretching open in response to an opening force. The central aperture diameter may be less than 30% of the resilient element diameter. The cuts may extend in a range of 65% to 95% of the diameter of the resilient element. The opening force may be provided by the holder. The opening force may be provided by the movement of prongs of the holder into the central aperture of the upstream end of the inhaler article when the inhaler article is inserted into the holder. Inserting the inhaler article into the holder may provide an opening force which forces the central aperture to stretch open in response to the opening force.

When the inhaler article is removed from the holder, the prongs are removed from the inhaler article and the resilient element closes. When the inhaler article is removed from the holder the opening force is removed. When the prongs are removed from the inhaler article, the opening force is removed. When the opening force is removed the resilient element closes. When the inhaler article is removed from the holder, the opening force is removed from the upstream end of the inhaler article. When the opening force is removed from the inhaler article, the resilient element returns to a closed position. Upon removal of the opening force, the resilient element closes to a closed position.

Between the downstream end and the upstream end of the tubular body is a central cavity which contains pharmaceutically active powder. The central cavity may have a length in a range from about 3 mm to about 12 mm, or from about 3 mm to about 7 mm or about 4 mm to about 6 mm, or about 5 mm.

The cavity may contain pharmaceutically active powder contained in a capsule. When the cavity contains pharmaceutically active powder contained in a capsule, the cavity is a capsule cavity.

The capsule cavity may be the inside of the inhaler article between the downstream blocker element and the upstream resilient element. The downstream blocker element may be joined with the upstream resilient element by a wrapper. The wrapper may form the tubular body of the inhaler article. The tubular body defining the capsule cavity may be formed of a biodegradable material, such as cardboard or paperboard.

The capsule cavity may have an inner diameter in a range from about 6 mm to about 7 mm, or about 6.5 mm to about 6.7 mm. The capsule cavity may have a lateral length in a range from about 15 mm to about 30 mm, or from about 20 mm to about 25 mm.

The capsule cavity may define a cylindrical space configured to contain a capsule (the capsule may have an obround shape or a circular cross-section, for example). The capsule cavity may have a substantially uniform or uniform diameter along the length of the capsule cavity. The capsule cavity may have a fixed cavity length. The capsule cavity has a cavity inner diameter, orthogonal to the longitudinal axis, and the capsule has a capsule outer diameter. The capsule cavity may be sized to contain an obround capsule. The capsule cavity may have a substantially cylindrical or cylindrical cross-section along the length of the capsule cavity. The capsule cavity may have a uniform inner diameter. The capsule may have an outer diameter that is about 80% to about 95% of the inner diameter of the capsule cavity. The configuration of the capsule cavity relative to the capsule may promote limited movement of the capsule during activation or piercing of the capsule.

The configuration of the capsule cavity relative to the capsule may promote the capsule to rotate with stability within the capsule cavity. The longitudinal axis of the capsule may rotate with stability co-axially with the longitudinal axis of the inhaler body during inhalation. The configuration of the capsule cavity relative to the capsule may promote the capsule to rotate with some shaking within the capsule cavity.

Stable rotation refers to the longitudinal axis of the inhaler body being substantially parallel or co-axial with the axis of rotation of the capsule. Stable rotation may refer to the absence of procession of the rotating capsule. Preferably the longitudinal axis of the inhaler body may be substantially coextensive with the axis of rotation of the capsule. Stable rotation of the capsule may provide a uniform entrainment of a portion of nicotine particles from the capsule over two or more, or five or more, or ten or more “puffs” or inhalations by a consumer.

The capsule may be contained within the inhaler article prior to consumption. The inhaler article may be contained within the capsule cavity by the resilient element.

The capsule may be formed of an airtight material that may be pierced or punctured by a piercing element that may be separate or combined with the inhaler. The capsule may be formed of a metallic or polymeric material that serves to keep contaminates out of the capsule but may be pierced or punctured by a piercing element prior to consumption of the nicotine particles within the capsule. The capsule may be formed of a polymer material. The polymer material may be hydroxypropylmethylcellulose (HPMC). The capsule may be a size 1 to size 4 capsule, or a size 3 capsule.

The capsule contains pharmaceutically active particles. The pharmaceutically active particles may comprise nicotine (also referred to as “nicotine powder” or “nicotine particles”) and optionally particles comprising flavour (also referred to as “flavour particles). The capsule may contain a predetermined amount of nicotine particles and optional flavour particles. The capsule may contain enough nicotine particles to provide at least 2 inhalations or “puffs”, or at least about 5 inhalations or “puffs”, or at least about 10 inhalations or “puffs”. The capsule may contain enough nicotine particles to provide from about 5 to about 50 inhalations or “puffs”, or from about 10 to about 30 inhalations or “puffs”. Each inhalation or “puff” may deliver from about 0.1 mg to about 3 mg of nicotine particles to the lungs of the user or from about 0.2 mg to about 2 mg of nicotine particles to the lungs of the user or about 1 mg of nicotine particles to the lungs of the user.

The nicotine particles may have any useful concentration of nicotine based on the particular formulation employed. The nicotine particles may have at least about 1% wt nicotine up to about 30% wt nicotine, or from about 2% wt to about 25% wt nicotine, or from about 3% wt to about 20% wt nicotine, or from about 4% wt to about 15% wt nicotine, or from about 5% wt to about 13% wt nicotine. Preferably, about 50 to about 150 micrograms of nicotine may be delivered to the lungs of the user with each inhalation or “puff”.

The capsule may hold or contain at least about 5 mg of nicotine particles or at least about 10 mg of nicotine particles. The capsule may hold or contain less than about 900 mg of nicotine particles, or less than about 300 mg of nicotine particles, or less than 150 mg of nicotine particles. The capsule may hold or contain from about 5 mg to about 300 mg of nicotine particles or from about 10 mg to about 200 mg of nicotine particles.

When flavour particles are blended or combined with the nicotine particles within the capsule, the flavour particles may be present in an amount that provides the desired flavour to each inhalation or “puff” delivered to the user.

The nicotine particles may have any useful size distribution for inhalation delivery preferentially into the lungs of a user. The capsule may include particles other than the nicotine particles. The nicotine particles and the other particles may form a powder system.

The capsule may hold or contain at least about 5 mg of a dry powder (also referred to as a powder system) or at least about 10 mg of a dry powder. The capsule may hold or contain less than about 900 mg of a dry powder, or less than about 300 mg of a dry powder, or less than about 150 mg of a dry powder. The capsule may hold or contain from about 5 mg to about 300 mg of a dry powder, or from about 10 mg to about 200 mg of a dry powder, or from about 25 mg to about 100 mg of a dry powder.

The dry powder or powder system may have at least about 40%, or at least about 60%, or at least about 80%, by weight of the powder system comprised in nicotine particles having a particle size of about 5 micrometers or less, or in a range from about 1 micrometer to about 5 micrometres.

The particles comprising nicotine may have a mass median aerodynamic diameter of about 5 micrometres or less, or in a range from about 0.5 micrometres to about 4 micrometres, or in a range from about 1 micrometre to about 3 micrometres or in a range from about 1.5 micrometres to about 2.5 micrometres. The mass median aerodynamic diameter is preferably measured with a cascade impactor.

The particles comprising flavour may have a mass median aerodynamic diameter of about 20 micrometres or greater, or about 50 micrometres or greater, or in a range from about 50 to about 200 micrometres, or from about 50 to about 150 micrometres. The mass median aerodynamic diameter is preferably measured with a cascade impactor.

The dry powder may have a mean diameter of about 60 micrometres or less, or in a range from about 1 micrometres to about 40 micrometres, or in a range from about 1.5 micrometres to about 25 micrometres. The mean diameter refers to the mean diameter per mass and is preferably measured by laser diffraction, laser diffusion or an electronic microscope.

The filter element may provide structure which allows particles having a desirable size to pass through, while preventing particles having larger size from passing through the filter. For example, the filter element may dry powder having a mean diameter from about 60 micrometres or less to pass through the filter element, while preventing dry powder having a mean diameter above about 60 micrometres from passing through. Or, if flavour particles are desired, the filter element may provide structure which allows particles having up to 200 micrometres to pass through the filter.

Nicotine in the powder system or nicotine particles may be a pharmaceutically acceptable free-base nicotine, or nicotine salt or nicotine salt hydrate. Useful nicotine salts or nicotine salt hydrates include nicotine pyruvate, nicotine citrate, nicotine aspartate, nicotine lactate, nicotine bitartrate, nicotine salicylate, nicotine fumarate, nicotine mono-pyruvate, nicotine glutamate or nicotine hydrochloride, for example. The compound combining with nicotine to form the salt or salt hydrate may be chosen based on its expected pharmacological effect.

The nicotine particles preferably include an amino acid. Preferably the amino acid may be leucine such as L-leucine. Providing an amino acid such as L-leucine with the particles comprising nicotine, may reduce adhesion forces of the particles comprising nicotine and may reduce attraction between nicotine particles and thus reduce agglomeration of nicotine particles. Similarly, adhesion forces to particles comprising flavour may also be reduced thus agglomeration of nicotine particles with flavour particles is also reduced. The powder system described herein thus may be a free-flowing material and possess a stable relative particle size of each powder component even when the nicotine particles and the flavour particles are combined.

The nicotine may be a surface modified nicotine salt where the nicotine salt particle comprises a coated or composite particle. A preferred coating or composite material may be L-leucine. One particularly useful nicotine particle may be nicotine bitartrate with L-leucine.

The powder system may include a population of flavour particles. The flavour particles may have any useful size distribution for inhalation delivery selectively into the mouth or buccal cavity of a user.

The powder system may have at least about 40%, or at least about 60%, or at least about 80%, by weight of the population of flavour particles of the powder system comprised in particles having a particle size of about 20 micrometres or greater. The powder system may have at least about 40% or at least about 60%, or at least about 80%, by weight of the population of flavour particles of the powder system comprised in particles having a particle size of about 50 micrometres or greater. The powder system may have at least about 40% or at least about 60%, or at least about 80%, by weight of the population of flavour particles of the powder system comprised in particles having a particle size in a range from about 50 micrometers to about 150 micrometres.

The particles comprising flavour may include a compound to reduce adhesion forces or surface energy and resulting agglomeration. The flavour particle may be surface modified with an adhesion reducing compound to form a coated flavour particle. One preferred adhesion reducing compound may be magnesium stearate. Providing an adhesion reducing compound such as magnesium stearate with the flavour particle, especially coating the flavour particle, may reduce adhesion forces of the particles comprising flavour and may reduce attraction between flavour particles and thus reduce agglomeration of flavour particles. Thus, agglomeration of flavour particles with nicotine particles may also be reduced. The powder system described herein thus may possess a stable relative particle size of the particles comprising nicotine and the particles comprising flavour even when the nicotine particles and the flavour particles are combined. The powder system preferably may be free flowing.

Conventional formulations for dry powder inhalation contain carrier particles that serve to increase the fluidization of the active particles since the active particles may be too small to be influenced by simple airflow though the inhaler. The powder system may comprise carrier particles. These carrier particles may be a saccharide such as lactose or mannitol that may have a particle size greater than about 50 micrometres. The carrier particles may be utilized to improve dose uniformity by acting as a diluent or bulking agent in a formulation.

The powder system utilized with the nicotine powder delivery system described herein may be carrier-free or substantially free of a saccharide such as lactose or mannitol. Being carrier-free or substantially free of a saccharide such as lactose or mannitol may allow the nicotine and to be inhaled and delivered to the user's lungs at inhalation or airflow rates that are similar to typical smoking regime inhalation or airflow rates.

The nicotine particles and a flavour may be combined in a single capsule. As described above, the nicotine particles and a flavour may each have reduced adhesion forces that result in a stable particle formulation where the particle size of each component does not substantially change when combined. Alternatively, the powder system includes nicotine particles contained within a single capsule and the flavour particles contained within a second capsule.

The nicotine particles and flavour particles may be combined in any useful relative amount so that the flavour particles are detected by the user when consumed with the nicotine particles. Preferably the nicotine particles and a flavour particles form at least about 90% wt or at least about 95% wt or at least about 99% wt or 100% wt of the total weight of the powder system.

A holder for an inhaler article includes a housing comprising a housing cavity for receiving an inhaler article, a moveable cap to enable engagement of the inhaler article with a piercing element, a piercing element, prongs to insert into the upstream end of the inhaler article when the inhaler article is inserted into the holder, and air inlets and airflow passages to enable swirling airflow through the inhaler article when it is engaged in the holder. This swirling or rotational inhalation airflow may be transmitted into an inhaler article to rotate and agitate a capsule and release dry powder contained within the capsule. The holder has an open end and a piercing end. The housing cavity is sized to receive an inhaler article. The inhaler article is inserted into the open end.

The holder has a piercing element. The piercing element is affixed to the inside of the bottom surface of the holder. The piercing element length may be any suitable length relative to the housing length. For example, the piercing element length may be about 25% to about 60%, or about 30% to about 50%, of the housing length. A distal end of the piercing element may be fixed to the distal end adjacent to or at the distal end of the housing. The piercing element entire length may be coextensive within the housing length. The piercing element is formed of a rigid material. The rigid material is sufficiently rigid to pierce, puncture or activate a capsule contained within the inhaler article. The piercing element may be formed of a metal. The piercing element may be formed of stainless steel, such as 316 stainless steel, for example. The piercing element may be formed of a polymeric material. The piercing element may be formed of a fibre-reinforced polymeric material.

The piercing element extends through the moveable cap. The moveable cap is moveable in relation to the piercing element. When the inhaler article is inserted into the holder, and is pressed down, the moveable cap moves down into the holder, exposing the piercing element. When the inhaler article is pressed down against the moveable cap, the piercing element extends into the inhaler article and pierces the capsule. Piercing the capsule activates the capsule. Piercing the capsule activates the inhaler system. Piercing the capsule allows for the release of powder from the capsule.

When the inhaler article is inserted into the holder, a portion of the holder inserts into the upstream end of the inhaler article to open the upstream end of the inhaler article. When a portion of the holder inserts into the upstream end of the inhaler article, the holder provides an opening force to the inhaler article. In embodiments, the portion of the holder that inserts into the upstream end of the inhaler article is prongs. When the inhaler article is inserted into the holder, prongs of the holder insert into the upstream end of the inhaler article. When prongs of the holder insert into the upstream end of the inhaler article, the prongs provide an opening force to the inhaler article. When the prongs of the holder insert into the upstream end of the inhaler article, the upstream end of the inhaler article is opened. When the prongs of the holder insert into the upstream end of the inhaler article, an opening force is applied to the upstream end of the inhaler article. When the prongs of the holder insert into the upstream end of the inhaler article which is a resilient element, the resilient element opens. When the inhaler article is removed from the holder, the opening force is removed from the upstream end of the inhaler article. When the opening force is removed from the inhaler article, the resilient element returns to a closed position. Upon removal of the opening force, the resilient element closes to a closed position.

When the inhaler article is inserted into the holder, a portion of the holder inserts into the resilient element at the upstream end of the inhaler article to open the resilient element the inhaler article. When a portion of the holder inserts into the resilient element of the inhaler article, the holder provides an opening force to the inhaler article. When a portion of the holder inserts into the resilient element of the inhaler article, the holder provides an opening force to the resilient element. When a portion of the holder inserts into the upstream end of the inhaler article, the holder provides an opening force to the upstream end of the inhaler article. When the inhaler article is removed from the holder, the opening force is removed from the upstream end of the inhaler article. When the opening force is removed from the inhaler article, the resilient element returns to a closed position. Upon removal of the opening force, the resilient element closes to a closed position.

Prongs can be shaped and sized to optimize insertion into the upstream end of the inhaler article. Prongs can be shaped and sized to optimize insertion into the resilient element of the inhaler article. For example, when the resilient element has a central aperture, the prongs may be angled to optimize insertion into the central aperture and expansion of the resilient element. Two or more prongs may be present. For example, 2, prongs may be present. Three prongs may be present. Four prongs may be present. Five prongs may be present. Six prongs may be present. More than six prongs may present. Or, prongs may mean an annular ring which inserts into the upstream end of the inhaler article. Or, prongs may mean an annular ring which inserts into the resilient element of the inhaler article.

The housing may be formed of any rigid material. The housing may be formed of a polymeric material. Polymeric materials useful for forming the housing include polycarbonate, polypropylene, polyethylene, nylon, acrylonitrile butadiene styrene, styrene acrylonitrile, polyacrylate, polystyrene, PBT polyester, PET polyester, polyoxymethylene, polysulfone, polyethersulfone, polyethereetherketone, or liquid crystal polymer.

The present disclosure provides an inhaler article having pharmaceutically active dry powder contained in a capsule to the lungs of a user. Disclosed herein is an inhaler article containing a capsule, the capsule containing pharmaceutically active powder. In embodiments, the pharmaceutically active powder contains nicotine, although other pharmaceutically active powders are contemplated in this disclosure, as discussed below. The inhaler article can be used to deliver pharmaceutically active powder to a user when the user inhales from the mouthpiece end (the downstream end, the proximal end) of the inhaler article. In order to deliver pharmaceutically active dry powder that is contained in a capsule to the user, the pharmaceutically active powder is released from the capsule, the powder is aerosolized, and inhaled by the user. That is, the powder is released from the capsule and entrained into the airflow created by the user when the user inhales from the mouthpiece end (the downstream end, the proximal end) of the inhaler article.

According to the present disclosure, the inhaler article has a tubular body extending along a longitudinal axis from a downstream mouthpiece end to an upstream end. Inside of the tubular body is a capsule cavity. The capsule cavity contains a capsule containing pharmaceutically active powder.

In order to release the pharmaceutically active powder from the capsule contained in the capsule cavity of the inhaler article, the capsule must open in a way that allows the powder contained inside the capsule to be released. In an embodiment, the capsule is pierced. When the capsule is pierced, a hole is introduced into the capsule. This releasing step or piercing step may also be considered an “activation”. According to the present disclosure, the capsule is activated when it is pierced by a piercing element. In embodiments, the piercing element is located in a holder.

When the inhaler article is inserted into a holder, the upstream end of the inhaler article is inserted into the holder. While inserting the inhaler article into the holder, the inhaler article may press down against a piercing element located in the holder, and the capsule may be pierced by the piercing element. Once inserted, the downstream end of the inhaler article extends from the holder and is accessible to the mouth of a user. Once the capsule has been pierced or activated, powder contained in the capsule can be released from the capsule. Powder from the capsule can then be released into an airflow and is inhaled by a user. The inhaler article is inserted into a holder to form an inhaler system, the capsule is pierced by a piercing element of the holder, an airflow is initiated by the user, the airflow passes through the inhaler article, entrains powder that has been released from the capsule, and the pharmaceutically active powder is delivered to a mouth of a user.

The holder is separate from the inhaler article, but the consumer may utilize both the inhaler article and the holder while consuming the particles released within the inhaler article. A plurality of these inhaler articles may be combined with a holder to form a system or kit. A single holder may be utilized on 10 or more, or 25 or more, or 50 or more, or 100 or more, inhaler articles to activate (puncture or pierce) a capsule contained within each inhaler article and provide reliable activation.

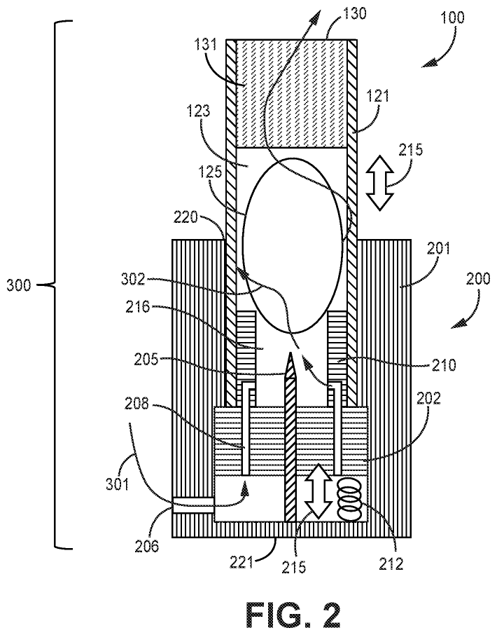

A holder for an inhaler article includes a housing comprising a housing cavity for receiving an inhaler article and configured to retain an inhaler article within the housing cavity. A moveable cap is in the housing cavity and is movable within the housing cavity along the longitudinal axis of the housing. The moveable cap has prongs. The prongs of the moveable cap are configured to insert into the inhaler article when the inhaler article is introduced into the holder. The moveable cap is configured to move in relation to a piercing element. When an inhaler article is introduced into the housing cavity of the holder, it is pressed down into the holder. As the inhaler article is pressed into the housing cavity, the prongs of the moveable cap insert into the upstream end of the inhaler article. At the same time, the inhaler article is pressed down into the housing against the moveable cap. The moveable cap moves down. The piercing element extends through the moveable cap as the moveable cap moves so that the capsule inside the inhaler article contacts and is pierced by the piercing element. The inhaler article is then released, and the moveable cap moves back to a resting position. The moveable cap may move back to a resting position by means of a spring. The position of the inhaler article, inserted into the holder, after the capsule has been pierced, is shown in FIG. 2 below.

The prongs of the moveable cap are able to insert into the upstream end of the inhaler article through the resilient element at the upstream end of the inhaler article.

A method includes, inserting an inhaler article into the housing cavity of the holder for an inhaler article, as described herein. The inhaler article includes a body, the body extending along an inhaler longitudinal axis from a mouthpiece end to a distal end, a body length, and a capsule disposed within the inhaler article body. Then, moving the inhaler article and sleeve toward the piercing element until the piercing element pierces the capsule. Then drawing air into the second opposing end of the housing cavity of the holder to form the swirling inhalation airflow. This swirling inhalation airflow is then transmitted into the inhaler article while the inhaler article is disposed within the holder for an inhaler article. The consumed inhaler article may then be removed from the holder. When the consumed inhaler is removed from the holder, the resilient element closes sufficiently to prevent the capsule from falling out of the spent inhaler article. The consumed inhaler article can be safely discarded. Then a fresh inhaler article may be inserted into the holder and the method may be repeated.

The inhaler article associated with the holder described above is configured to receive swirling inhalation airflow directly into the distal end of the inhaler article. The swirling inhalation airflow is initiated when a user “puffs” on the downstream end of the inhaler article inserted into a holder. This creates negative pressure inside the inhaler article. As a result of this negative pressure, air enters the holder via an air inlet. Air then travels through air passages in the holder. The air passages in the holder direct air into the inhaler article at an angle tangential to the longitudinal axis of the inhaler article, when inserted into the holder. This tangential air creates swirling airflow in the inhaler article when inserted into the holder. This swirling airflow causes the capsule to rotate and/or agitate within the inhaler article. This agitation of the capsule improves the flow of powder out of the activated capsule and improves the efficiency with which powder is entrained into airflow delivered to the user.

The resilient element may be made from any material suitable for opening and closing. For example, the resilient element may be made from silicon. The resilient element may be made from latex. The resilient element may be made from rubber. The resilient element may be made from plastic. The resilient element may be made from paper. The resilient element may be made from aluminium foil. The resilient element may be made from laminated layered PLA on a paper layer. The resilient element may be made from cardboard. The resilient element may be made from silicon, latex or rubber. The resilient element may be made from silicon, latex, rubber of a combination thereof.

The resilient element may be affixed to the inhaler article. The resilient element may be affixed to the inhaler article by any means. For example, the resilient element may be affixed to the inhaler article by gluing, friction fitting, heat sealing, or by any means.

Resilient material may be cut into disks from a ribbon of material suitable for forming the resilient element. The ribbon of resilient material may be fed from a feeder reel to a take-up reel across a cutting stage. Disks which will form resilient elements may be cut by a cutter at the cutting stage. In addition, cuts may be made to form resilient elements having cuts and flaps, or central apertures may be cut in the resilient element disks, or both cuts and central apertures may be cut into the resilient element at this cutting stage.

Cut disks may be transported to a gluing station by a holder. Glue may be introduced to a gluing stage. For example, glue may be applied to the stage. Glue may be pressure-fed to glue rings, sized slightly smaller than the diameter of resilient element disks at the glue stage. Glue may be applied to the cut disks by presenting the cut disks to glue rings at the glue stage. Then, cut disks with applied glue may be applied to the upstream end of an inhaler article. The glue may be any glue known in the art. For example, glue may be water-based glue or hot melt glue.

The invention is defined in the claims. However, below there is provided a non-exhaustive list of non-limiting examples. Any one or more of the features of these examples may be combined with any one or more features of another example, embodiment, or aspect described herein.
Example Ex1: A method for manufacturing an inhaler article having an upstream resilient element for insertion into a holder to form an inhaler system for providing a dry powder to the lungs of a user, the manufacturing method comprising: moving a ribbon of resilient material from a feeder reel to a cutting stage; presenting the ribbon of resilient material to the cutting stage; cutting at least one disk of resilient material from the ribbon of resilient material to form at least one cut disk of resilient material; presenting the at least one cut disk of resilient material to a glue stage; dispensing at least one glue ring; applying the at least one glue ring to the at least one cut disk of resilient material; and, presenting the at least one cut disk of resilient material having an applied glue ring to an upstream end of an inhaler article; affixing the at least one cut disk of resilient material having an applied glue ring to the upstream end of an inhaler article to form an inhaler article having an affixed upstream resilient element.
Example Ex 2: The method of Example Ex1 wherein the cutting step further comprises cutting at least 2 cuts in the at least one disk of resilient material to form at least 4 flaps in each cut disk.
Example Ex 3: The method of Example Ex 1 wherein the cutting step further comprises cutting at least 3 cuts in the at least one disk of resilient material to form at least 6 flaps in each cut disk.
Example Ex 4: The method of Example Ex 1 wherein the cutting step further comprises cutting at least 4 cuts in the at least one disk of resilient material to form at least 8 flaps in each cut disk.
Example Ex 5: The inhaler article of any one of Examples Ex 2-Ex4 wherein the cuts extend in a range from about 65% to about 95% of a diameter of the resilient element.
Example Ex 6: The method of any one of the preceding Examples further comprising cutting a central aperture in the at least one disk of resilient material.
Example Ex 7: The method of Example Ex 6 wherein the central aperture has a diameter which comprises less than 30% of a diameter of the resilient element.
Example Ex 8: The method according to any one of the preceding Examples further comprising moving the ribbon of resilient material from the cutting stage to a take-up reel.
Example Ex 9: The method of any one of the preceding Examples further comprising the step of inserting a capsule into the inhaler article prior to the affixing step.
Example Ex 10: The method of any preceding Examples, wherein the resilient element is made from silicon, latex, plastic, paper, aluminium foil, paper tape, laminated layered material such as a PLA layer on a paper layer, or cardboard.
Example Ex 11: The method of any one of the preceding Examples wherein the resilient element comprises rubber, silicon or latex.
Example Ex 12: The method of any one of the preceding Examples wherein the inhaler article comprises: a tubular body extending along a longitudinal axis from a downstream mouthpiece end to an upstream end; a capsule cavity within the tubular body between the downstream mouthpiece end and the upstream end; the capsule cavity containing a capsule; wherein an upstream boundary of the capsule cavity is defined by the resilient element, and wherein the affixed resilient element retains the capsule in the capsule cavity.
Example Ex 13: The method of Example Ex 12 wherein the downstream mouthpiece end comprises a blocker element.
Example Ex 14: The method of any one of the preceding Examples wherein the tubular body comprises cardboard.
Example Ex 15: The method of any one of Examples 12-14 wherein the capsule contains pharmaceutically active dry powder comprising nicotine.
Example Ex16: The method of any one of the preceding Examples wherein the resilient material comprises color.

Examples will now be further described with reference to the figures in which:

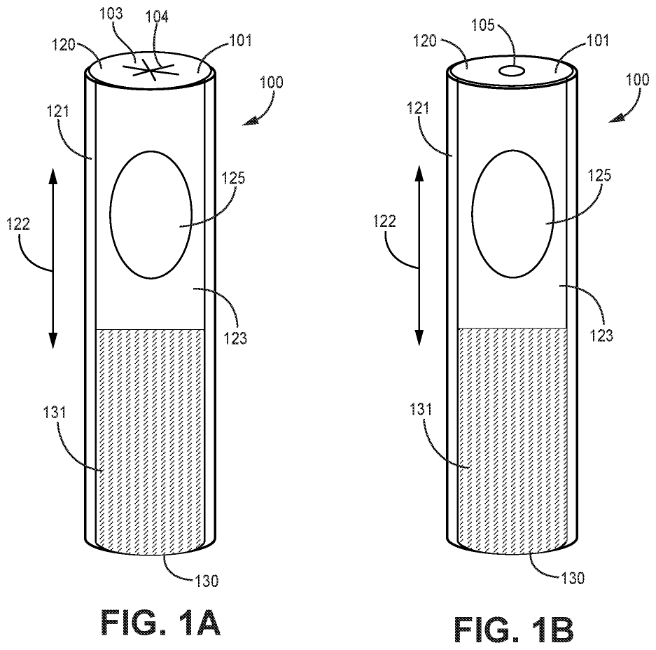

FIG. 1A and FIG. 1B show embodiments of the inhaler article of the present disclosure. FIG. 1A shows an embodiment of the inhaler article which has a resilient element at the upstream end which is cut to form flaps. FIG. 1B shows an embodiment of the inhaler article which has a resilient element at the upstream end, the resilient element having a central aperture.

FIG. 2 shows an inhaler article of the present disclosure inserted into a holder forming an inhaler system.

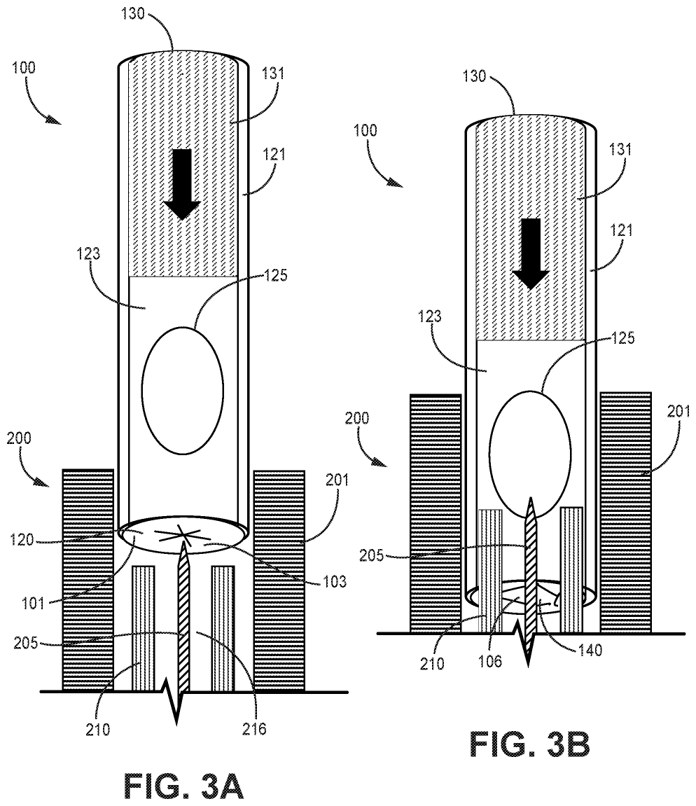

FIG. 3A and FIG. 3B illustrate the insertion of an inhaler article of the present disclosure into a holder. FIG. 3A illustrates an inhaler article which has a resilient element at the upstream end, cut to form flaps. FIG. 3B illustrates the inhaler article having a resilient element at the upstream end, cut to form flaps, inserted into a holder, where the flaps have been opened by the holder.

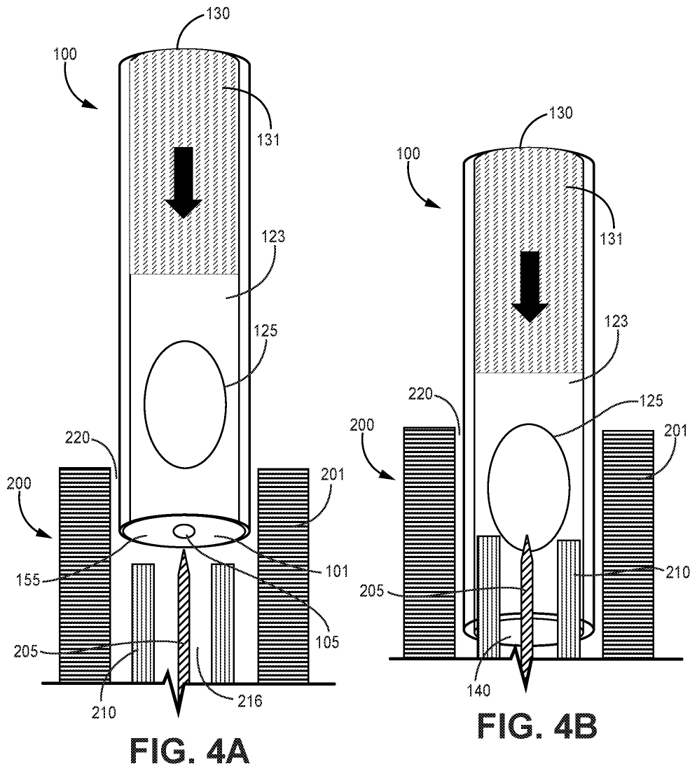

FIG. 4A and FIG. 4B illustrate the insertion of an inhaler article of the present disclosure into a holder. FIG. 4A illustrates an inhaler article which has a resilient element at the upstream end, the resilient element having a central aperture. FIG. 4B illustrates the inhaler article having a resilient element at the upstream end, having a central aperture, inserted into a holder, where the central aperture has been opened by the holder.

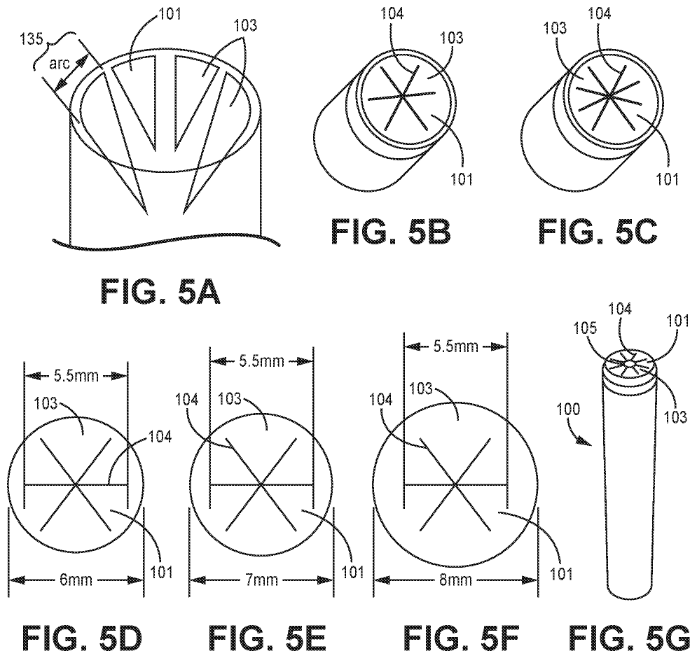

FIG. 5A, FIG. 5B, FIG. 5C, FIG. 5D, FIG. 5E, FIG. 5F and FIG. 5G illustrate embodiments of the inhaler article of the present disclosure having a resilient element at the upstream end, cut to form flaps. FIG. 5A illustrates the flaps folding back to form an internal opening. FIG. 5B illustrates an embodiment of the inhaler article of the present disclosure having a resilient element at the upstream end, cut to form flaps, having 6 radial cuts, to form 6 flaps. FIG. 5C illustrates an embodiment of the inhaler article of the present disclosure having a resilient element at the upstream end, cut to form flaps, having 8 radial cuts, to form 8 flaps. FIG. 5D illustrates an embodiment of the inhaler article of the present disclosure having a resilient element at the upstream end, cut to form flaps, having 6 radial cuts, to form 6 flaps illustrating, in an embodiment, a ratio of the length of the cuts in relation to the diameter of the inhaler article. FIG. 5E illustrates an embodiment of the inhaler article of the present disclosure having a resilient element at the upstream end, cut to form flaps, having 6 radial cuts, to form 6 flaps illustrating, in an embodiment, a ratio of the length of the cuts in relation to the diameter of the inhaler article. FIG. 5F illustrates an embodiment of the inhaler article of the present disclosure having a resilient element at the upstream end, cut to form flaps, having 6 radial cuts, to form 6 flaps illustrating, in an embodiment, a ratio of the length of the cuts in relation to the diameter of the inhaler article. FIG. 5G is an illustration of an embodiment of the inhaler article having a central aperture and flaps.

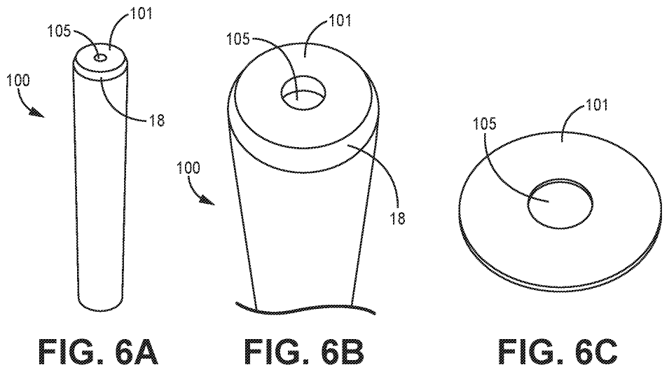

FIG. 6A, FIG. 6B and FIG. 6C are illustrations embodiments of the inhaler article of the present disclosure having a resilient element at the upstream end, where the resilient element has a central aperture. FIG. 6A is an illustration of the central aperture of the resilient element at the upstream end of the inhaler article. FIG. 6B is another illustration of the inhaler article of the present disclosure having a resilient element at the upstream end, where the resilient element has a central aperture. FIG. 6C is an illustration of the resilient element 101 having a central aperture 105.

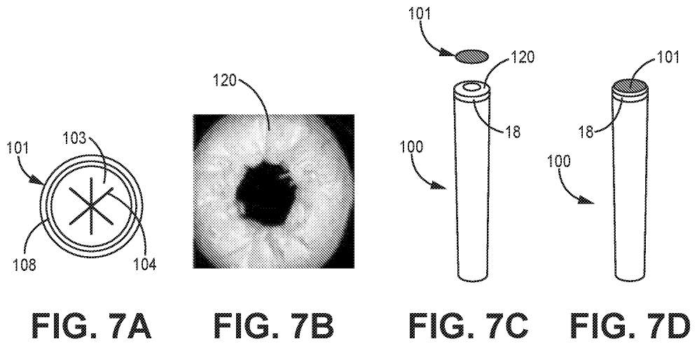

FIG. 7A, FIG. 7B FIG. 7C and FIG. 7D are illustrations of embodiments of the inhaler article 100 of the present disclosure. FIG. 7A is an illustration of the resilient element 101, having an applied ring of glue 108, prior to affixing the resilient element 101 to the upstream end 120 of the inhaler article 100. FIG. 7C is a photograph of an embodiment of the upstream end 120 of the inhaler article 100 before the resilient element 101 is affixed. FIG. 7C is an illustration of the application of a resilient element 101 to the upstream end 120 of the inhaler article 100. FIG. 7D shows an illustration of the inhaler article 100 after the resilient element has been affixed to the upstream end 120 of the inhaler article 100.

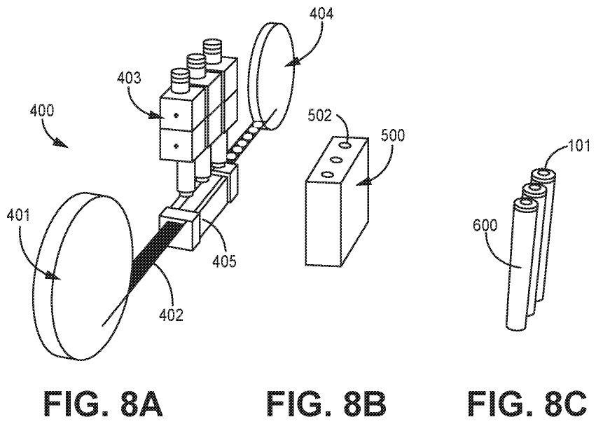

FIG. 8A, FIG. 8B and FIG. 8C illustrate manufacturing equipment for manufacturing embodiments of the inhaler article having a resilient element affixed to the upstream end of the inhaler article. FIG. 8A illustrates a ribbon cutter to cut disks of resilient material to create resilient elements. FIG. 8B shows a stage to provide a ring of glue in the manufacturing process. FIG. 8C shows a holder containing three inhaler articles, which will be presented to the resilient elements having an applied glue ring, during the manufacturing process.

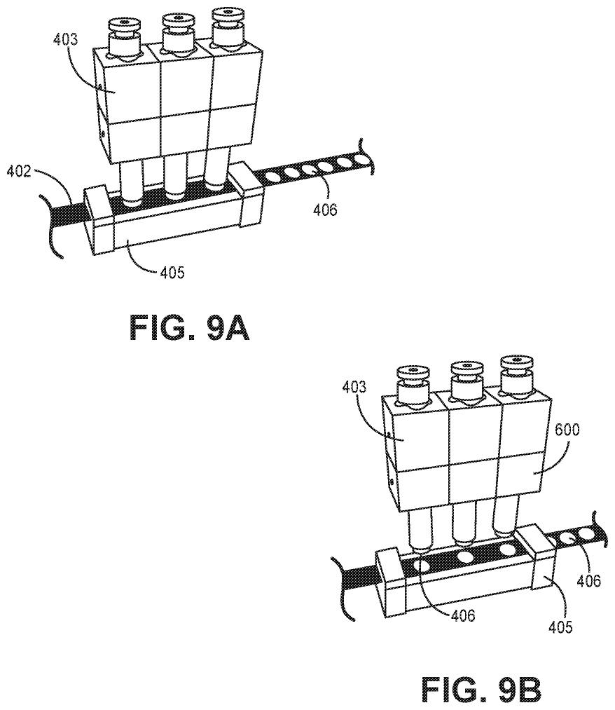

FIG. 9A and FIG. 9B illustrate manufacturing equipment for manufacturing embodiments of the inhaler article having a resilient element affixed to the upstream end of the inhaler article. FIG. 9A shows the ribbon cutter before resilient elements of resilient material have been cut from a ribbon of resilient material. FIG. 9B shows the ribbon cutter after resilient elements, disks of resilient material, have been cut from a ribbon of resilient material.

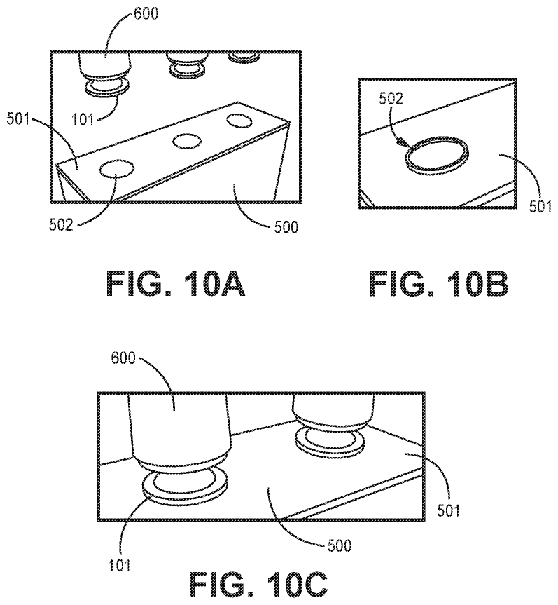

FIG. 10A, FIG. 10B, and FIG. 10C illustrate manufacturing equipment and methods for affixing the resilient element to the inhaler article in embodiments of the inhaler article having a resilient element affixed to the upstream end of the inhaler article. FIG. 10A illustrates a step of presenting the resilient element to the gluing station. FIG. 10B illustrates the step of loading the glue ring. FIG. 10C illustrates the step of applying glue to the resilient element.

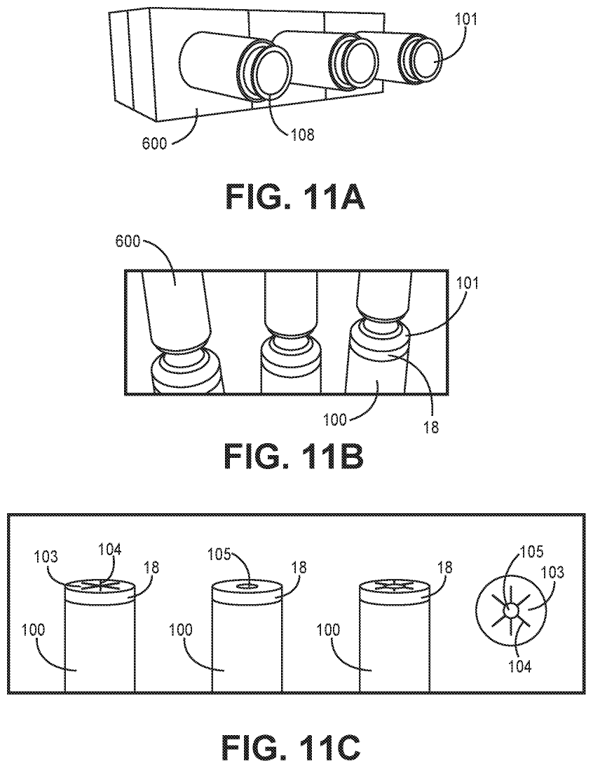

FIG. 11A, FIG. 11B, and FIG. 11C illustrate manufacturing equipment for manufacturing embodiments of the inhaler article having a resilient element affixed to the upstream end of the inhaler article. FIG. 11A illustrates a perspective view of the resilient element having an applied ring of glue, after the manufacturing step of FIG. 10. FIG. 11B illustrates the presentation of the inhaler article to the glue-side of the resilient element having an applied ring of glue. FIG. 11C illustrates embodiments of the inhaler article having a resilient element affixed to the upstream end of the inhaler article.

The schematic drawings are not necessarily to scale and are presented for purposes of illustration and not limitation. The drawings depict one or more aspects described in this disclosure. However, it will be understood that other aspects not depicted in the drawing fall within the scope and spirit of this disclosure.

FIG. 1A and FIG. 1B show embodiments of the inhaler article 100 of the present disclosure. FIG. 1A illustrates an inhaler article 100 having an upstream end 120 (also a distal end), a downstream end 130 (also a proximal end or a mouthpiece end), a tubular body 121 extending along a longitudinal axis 122 from the downstream end 130 to an upstream end 120. FIG. 1A shows an embodiment of the inhaler article which has a resilient element 101 at the upstream end 120 which is cut 104 to form flaps 103. FIG. 1A shows the cuts 104 and flaps 103 formed by the cuts 104. FIG. 1A and FIG. 1B also show the capsule cavity 123 containing a capsule 125. FIG. 1B shows an embodiment of the inhaler article which has a resilient element 101 at the upstream end 120, the resilient element having a central aperture 105. FIG. 1A and FIG. 1B also show that the inhaler article 100 may have a blocker 131 located near the downstream end 130 of the inhaler article 100.

The tubular body 121 may be made of a carton or wrapping paper rolled in a tube form. The resilient element 101 has an opening, formed, as shown in FIG. 1A, by cuts 104 forming flaps 103. These cut flaps 103 provide an opening in the resilient element 101. In FIG. 1B, the central aperture 105 is shown. These openings provide access for a piercing pin 205 to be able to reach and perforate the capsule 125 when the inhaler article 100 is placed in the holder 200 and the capsule 125 is activated. At the same time, these openings are smaller than the diameter of the capsule 125, to prevent the capsule 125 from falling out of the inhaler article 100 before, during or after use.

FIG. 2 shows an inhaler article 100 of the present disclosure inserted into a holder 200 forming an inhaler system 300. The inhaler article 100 has a capsule 125, a capsule cavity 123 and a tubular body 121. The holder 200 has a housing 201 defining a housing cavity 216. The housing cavity 216 has an open end 220 and a piercing end 221. When the inhaler article 100 is introduced into the holder 200, into the housing cavity 216, the inhaler article 100 pushes against the moveable cap 202 at the distal end of the housing cavity 216. The moveable cap 202 has prongs 210. These prongs 210 may be part of the moveable cap or may separate from the moveable cap. The prongs 210 are located in the housing cavity 216. Prongs 210 are structured to insert into the tubular body 121 of the inhaler article 100 when the inhaler article 100 is inserted into the holder 200. The moveable cap 202 moves down relative to the housing 201. When the inhaler article and the moveable cap 202 move down in relation to the housing 201, the piercing pin 205 extends through the resilient element (not shown in FIG. 2 but see FIG. 3A and FIG. 3B) and pierces the capsule 125.

There is an air inlet 206 through the housing 201 of the holder 200 which allows air to enter the inhaler system 300. Air flows in an air flow path 301 into the inhaler system 300 through the air inlet 206, through the airflow passage 208 through the moveable cap 202, into the capsule cavity 123 of the inhaler article 100. Because the airflow passage 208 is tangential to the longitudinal axis 122 of the inhaler article 100 (and the capsule cavity 123), air flowing through the capsule cavity 123 flows in a swirling airflow path 302. In embodiments, there is one airflow passage 208. In embodiments, there are two airflow passages 208. In embodiments, there are more than two airflow passages 208.

When using such an article, the user will insert the inhaler article 100 into the holder 200 to form an inhaler system 300. When inserting the inhaler article 100 into the holder 200, the user presses down on the inhaler article which moves the moveable cap 202 in relation to the piercing pin 205, and moves the capsule 125, contained in the capsule cavity 123 of the inhaler article, to contact the piercing pin 205, piercing the capsule 125. Once the capsule 125 has been pierced, the moveable cap 202 retracts, by the action of a spring 212, for example. The movement of the moveable cap is indicated by arrows 215.

After the capsule 125 is pierced, when this air flows into the system via the air inlet 206, through the airflow passages 208, through the capsule cavity 123, and out of the system to a user via the downstream end 130 of the inhaler article 100, powder released from the pierced capsule 125 is entrained into the airflow path 302 and powder is delivered to the downstream end 130 (the mouthpiece end) of the inhaler article 100, and is inhaled by the user. This airflow is initiated by suction provided by the user at the mouthpiece end, the downstream end 130 of the inhaler article 100. In addition, the swirling airflow path 302 provides agitating or swirling airflow which agitates the capsule 125 inside the capsule cavity 123 and improves release of powder from the capsule 125 during use.

It may be desirable to be able to create an effective air flow through the inhaler system 300 to entrain an appropriate amount of powder in the airflow so as to provide an appropriate dose of powder to a user. The powder is released from the capsule 125 through a hole introduced into the capsule by the piercing pin 205. This piercing pin 205 also passes through the upstream end 120 of the inhaler article. If the upstream end 120 of the inhaler article 100 is closed, the piercing pin 205 introduces a hole in the upstream end 120 of the inhaler article 100. The hole made by the piercing pin 205 in the upstream end 120 of an inhaler article is generally related to the size of the piercing pin 205. In general, a hole made by a piercing pin 205 is not large enough to enable sufficient airflow through the inhaler system 300 to provide an appropriate dose of powder to the user. That is, a small hole in the upstream end 120 of the inhaler article 100 in an inhaler system 300 introduces significant resistance to draw (RTD) into the system and creates a system that does not have sufficient airflow to provide an appropriate dose of powder released from a capsule 125 to a user. One solution to this RTD challenge is to provide an inhaler article 100 having an upstream end 120 that can open. A solution to this RTD challenge is to provide an inhaler article 100 having a resilient element 101 on the upstream end 120 of the inhaler article 100 that is opened by an opening force provided by a structure of the holder, for example prongs 210, to create an open airflow area to improve RTD through the system.

After the inhaler system 300 has been used, the user can withdraw the inhaler article 100 from the holder 200. After the inhaler article 100 is withdrawn from the holder, the resilient element 101 reverts back to its pre-use state, or nearly its pre-use state. That is, the resilient element 101 is opened to an open position in response to an opening force, and upon removal of the opening force, closes to a closed position. When the resilient element is in the closed position, the resilient element is sufficiently closed to retain the capsule in the capsule cavity between the downstream mouthpiece end and the resilient element.

It may be desirable to ensure that the capsule 125, containing active ingredient is retained in the inhaler article before and after insertion of the inhaler article 100 into the holder 200. In addition, it may be desirable to prevent powder from spilling from the inhaler article 100 before and after insertion of the inhaler article 100 into the holder 200. It may be desirable to prevent the capsule powder from falling out of or spilling from the inhaler article 100 before and after use. Providing an inhaler article 100 having an upstream end 120 that is open does not retain the capsule 125 inside the capsule cavity 123 of the inhaler article 100. The present disclosure provides solutions that address both the RTD challenge and the retention challenge. The solutions are resilient elements at the upstream end of the inhaler article, which can be closed (or relatively closed) to retain the capsule and powder before and after insertion into a holder 200 to form an inhaler system 300, and opened during use, during insertion into a holder 200, to provide appropriate airflow and an appropriate dose of powder to a user of the inhaler system 300.

In addition, providing a resilient element 101 applied or affixed to the upstream end of the inhaler article 100, where the resilient element 101 is affixed sufficiently to prevent easy removal of the resilient element 101 is desirable.

As shown in FIG. 2, when inserting the inhaler article into the holder the user simultaneously moves the moveable cap 202, perforates the capsule 125, and inserts the insertion portion of the moveable cap 202 into the inhaler article to provide an airflow opening at the upstream end 120 of the inhaler article 100 which provides an appropriate RTD for the proper operation of the inhaler system 300. That is, the hole at the upstream end 120 of the inhaler article 100 is large enough for the inhaler system to function, when the insertion portion of the moveable cap is inserted into the inhaler article, and the resilient element 101 is moved or stretched open.

In addition, when the inhaler article 100 is removed from the holder 200, the resilient element reverts to its pre-insertion state (partially or fully). When the resilient element 101 reverts to its pre-insertion state, the capsule 125 and the powder are contained inside the inhaler article 100.

Prongs 210 are illustrated as pillars that insert into the inhaler article in FIG. 3. Two or more prongs 210 may be present. For example, there may be two prongs 210. There may be three prongs 210. There may be four prongs 210. There may be five prongs 210. There may be six or more prongs 210. Prongs 210 may be an annular ring structure. Prongs 210 may be a conical structure to insert into a central aperture to stretch the central aperture open. Prongs 210 may be any suitable shape or size to open the resilient element of the inhaler article.

FIG. 3A and FIG. 3B illustrate the insertion of an inhaler article 100 having a resilient element 101 having cut flaps 103 into the holder 200. The arrows in FIG. 3A and FIG. 3B illustrate the insertion of the inhaler article 100 into the holder 200. FIG. 3A illustrates an inhaler article which has a resilient element 101 at the upstream end 120, cut to form flaps 103. FIG. 3B illustrates the inhaler article 100 having a resilient element 101 at the upstream end 120, cut to form flaps 103, inserted into a holder 200, where the flaps 103 have been opened by the prongs 210 of the moveable cap (not shown) of the holder 200. FIG. 3B illustrates that when the inhaler article 100 has been inserted into the holder 200, the pin 205 pierces the capsule 125 in the capsule cavity 123 of the inhaler article 100.

After the inhaler system 300 has been used, the user can withdraw the inhaler article 100 from the holder 200. After the inhaler article 100 is withdrawn from the holder, and the insertion portion of the moveable cap 210 is removed from the inhaler article 100, the resilient element 101 reverts back to its pre-use state, or nearly its pre-use state. After use, the flaps 103 on the upstream end 120 of the inhaler article 100 revert back to a closed state, as shown in, for example, FIG. 1A.

FIG. 4A and FIG. 4B illustrate the insertion of an inhaler article 100 of the present disclosure into a holder 200 to form an inhaler system 300. The arrows in FIG. 4A and FIG. 4B illustrate the insertion of the inhaler article 100 into the holder 200. FIG. 4A illustrates an inhaler article 100 which has a resilient element 101 at the upstream end 120, the resilient element 101 having a central aperture 105. FIG. 4B illustrates the inhaler article 100 having a resilient element 101 at the upstream end 120, having a central aperture 105, inserted into a holder 200, where the central aperture 105 has been opened to form an airflow aperture 140. The central aperture 105 has been opened by an opening force provided by prongs 210 to form an airflow aperture by inserting the insertion portion of the moveable cap 210 through the central aperture 105 of the resilient element 101 of the inhaler article 100.

After the inhaler system 300 has been used, the user can withdraw the inhaler article 100 from the holder 200. After the inhaler article 100 is withdrawn from the holder, and the insertion prongs 210 of the moveable cap (not shown) is removed from the inhaler article 100, the resilient element 101 reverts back to its pre-use state, or nearly its pre-use state. After use, the central aperture 105 of the resilient element 101 reverts back to a closed state, as shown in, for example FIG. 1B.

FIG. 5A, FIG. 5B, FIG. 5C, FIG. 5D, FIG. 5E, FIG. 5F and FIG. 5G illustrate embodiments of the inhaler article of the present disclosure having a resilient element 101 at the upstream end, cut to form flaps 103. The number of cuts 104 and length of the cuts 104 define the geometry of the flaps 103 that are formed by the cuts 104. FIG. 5A illustrates the flaps 103 folding back to form an internal opening. FIG. 5A shows that the number of cuts 104 define the arc 135 and the shape of the flap 103 where the flaps fold inward. The number of cuts (3 diametric cuts or 6 radial cuts as shown in FIG. 5B or 8 radial cuts or 4 diametric cuts as shown in FIG. 5C) changes the arc 135. Increasing the number of cuts reduces the arc 135. For example, where there are two diametric cuts, four flaps would be formed. For example, where there are 3 diametric cuts or 6 radial cuts as shown in FIG. 5B, six flaps are formed, the arc 135 is 3.14 mm in an inhaler article having a diameter of 10 mm. Where there are 4 diametric cuts or 8 radial cuts as shown in FIG. 5C, eight flaps are formed, the arc 135 is 2.34 mm in an inhaler article having a diameter of 10 mm. Increasing the number of cuts 104, and therefore the number of flaps 103, reduces the arc 135. In addition, the length of the cuts 104 affects the arc 135. Changing the geometry of these flaps, and changing the arc 135, may affect the degree to which the flaps can open during use. In addition, adjusting the length of the cuts 104 and the number of flaps 103 may enable different materials to be used for the resilient element 101. For example, providing more flaps may allow the elastic material to be less elastic, and still rebound to its original shape, or nearly its original shape, after opening the flaps 103. In embodiments, the elastic material may be, for example, silicon, latex, rubber, plastic, paper, aluminium foil, paper tape, laminated layered PLA on a paper layer or cardboard. Where more elasticity is required, the material may be selected from silicon, latex, rubber or a combination. In embodiments, the resilient element can be, for example, less than 0.5 mm in thickness.

Further, as shown in FIGS. 5D, 5E and 5F, the length of the cuts may also affect the size and shape of the flaps and the arc 135. The size of the flaps 103, the length of the cuts 104 and the geometry of the arc 135 may be adjusted, in correlation with the material of the resilient element, to optimize the ability of the resilient element to be closed before and after use, and to open to form a suitable airflow pathway during use when the inhaler article 100 is inserted into the holder 200 to form the inhaler system 300. FIGS. 5D, 5 and 5F illustrate cuts 104 which form flaps 103, where the cuts extend about 90% of the diameter of the inhaler article (FIG. 5D), about 78% of the diameter of the inhaler article (FIG. 5E) and about 65% of the inhaler article (FIG. 5F). FIG. 5G is a photograph of an embodiment of the inhaler article of the present disclosure having a resilient element at the upstream end, pre-cut to form flaps, having 6 radial cuts, to form 6 flaps. FIG. 5G is an illustration of an embodiment of the inhaler article 100 having a central aperture 105 and cuts 104 that form flaps 103. In embodiments, the resilient element 101 is cut to form at least 4 flaps. In embodiments, the resilient element 101 is cut to form at least 6 flaps. In embodiments, the resilient element 101 is cut to form 4, 6 or 8 flaps. Such number of flaps provides for the arcs areas of the resilient element to provide good flexibility and resistance to tearing ((when pushed into the holder 200), as well as an appropriate rigidity to come back to its initial position when the article is removed from the holder 200. In embodiments, the cuts extend about 65% to 95% of the diameter of the resilient element 101.

In embodiments, the cuts have the same length and cross at the center of the resilient element and have similar angles between them providing symmetrical distribution of a force exerted on the flaps when pushed into the holder 200. Providing this symmetry may assist with preventing tearing of the resilient element upon inserting the inhaler article into the holder and may contribute to the desired opening and closing of the resilient elements.

FIG. 6A, FIG. 6B and FIG. 6C are illustrations embodiments of the inhaler article of the present disclosure having a resilient element 101 at the upstream end, where the resilient element 101 has a central aperture 105. FIG. 6A is an illustration of the central aperture 105 of the resilient element 101 applied on an upstream element 18 at the upstream end 120 of the inhaler article 100. FIG. 6B is another illustration of the inhaler article 100 of the present disclosure having a resilient element 101 at the upstream end 120, where the resilient element has a central aperture 105. In embodiments, the resilient element 101 is applied on an upstream element 18. The upstream element 18 may be present or absent. FIG. 6C is an illustration of the resilient element 101 having a central aperture 105 alone. In additional embodiments, the resilient element may have a combination of a central aperture and flaps. For example, the central aperture may be partly cut or weakened. In embodiments, the diameter of the central hole may be about 30% of the disc diameter, less than 30%.

FIG. 7A, FIG. 7B FIG. 7C and FIG. 7D are illustrations of embodiments of the inhaler article 100 of the present disclosure. FIG. 7 is an illustration of the resilient element 101, having an applied ring of glue 108, prior to affixing the resilient element 101 to the upstream end 120 of the inhaler article 100. FIG. 7B is a photograph of an embodiment of the upstream end 120 of the inhaler article 100 before the resilient element 101 is affixed. The upstream end 120 of the inhaler article may be an upstream element 18 or may be the upstream end 120 of the tubular body 121. FIG. 7C is an illustration of the application of a resilient element 101 to the upstream end 120 of the inhaler article 100. FIG. 7D shows an illustration of the inhaler article 100 after the resilient element has been affixed to the upstream end 120 of the inhaler article 100. In embodiments, the resilient element may have indicia. For example, the resilient element may be colored. Or, the resilient element may have one or more symbols. Or, the resilient element may have both color and one or more symbols.

FIG. 8A, FIG. 8B and FIG. 8C illustrate manufacturing equipment 400 and methods for manufacturing embodiments of the inhaler article 100 having a resilient element 101 affixed to the upstream end 120 of the inhaler article 100. This manufacturing equipment 400 operates to cut out round disks of resilient material to form resilient elements 101 that fit at the upstream end 120 of the inhaler article 100. FIG. 8A shows a ribbon cutter 403 which has a feeder reel 401, a ribbon of resilient material 402, a cutter 403, a cutting stage 405 and a take-up reel 404. In use, as shown in FIG. 8A, the feeder reel 401 contains an uncut ribbon of resilient material 402. This uncut ribbon of resilient material 402 is moved from the feeder reel 401 to the cutting stage 405. The ribbon of resilient material 402 is presented to the cutting stage 405. The cutting stage 405 is aligned with a ribbon cutter 403. The cutter 403 cuts round disks of resilient material to create resilient elements 101. In addition, the cutter 403 may make cuts in the resilient elements to form flaps 103, making a resilient element 101 having flaps 103. Or, the cutter 403 may cut a central aperture 105 in the disk to form resilient elements 101 having a central aperture 105. Alternatively, cuts 104 or central apertures 105 may be made in the resilient elements 101 in a separate cutting step. Or, the cutter 403 may make cuts 104 and a central aperture 105 in the same cutting action. After the disks are cut from the ribbon 402, the used ribbon 406 proceeds to a take-up reel 404. FIG. 8B shows a glue station 500 having a glue stage 501 to optionally provide a ring of glue to the disks or resilient elements in the manufacturing process FIG. 8C shows an element holder 600 holding three resilient elements which, in use, are presented to the glue stage 502 to apply glue to the cut resilient elements during the manufacturing process. Or, stated another way, the at least one cut disk of resilient material having an applied glue ring is presented to an upstream end of an inhaler article.

FIG. 9A and FIG. 9B illustrate manufacturing equipment and methods for manufacturing embodiments of the inhaler article 100 having a resilient element 101 affixed to the upstream end 120 of the inhaler article 100. FIG. 9A shows the cutting step wherein a ribbon 402 of resilient material is moved from the feeder reel 401 to the cutting stage 405. The ribbon of resilient material is presented to a cutting stage 405, and the ribbon cutter 403 addresses the cutting stage 405 with the ribbon 402 of resilient material. The cutter cuts at least one disk of resilient material from the ribbon of resilient material to form at least one cut disk of resilient material. As shown in FIG. 9A and FIG. 9B, three cutters 403 are present. The cutters 403 are modular and may be combined to optimize the manufacture process. In embodiments, one cutter 403 may be present. In embodiments two cutters 403 may be present. In embodiments, three cutters 403 may be present. In embodiments, more than three cutters 403 may be present. The ribbon cutter 403 cuts disks of resilient material from a ribbon 402 of resilient material. The cutting may occur by press-cutting, knife-cutting, laser cutting, or by any means. FIG. 9B shows step of removing the disks of resilient material from the cutting stage 405. Once the cut has been made, the disks of resilient material may be removed from the cutting stage 405 to proceed to the next step in the manufacturing process. In addition, the cut ribbon 406 proceeds to the take-up reel and fresh, un-cut ribbon will be presented to the cutting stage 405 so that the cutting step can be repeated. The cutting step may also include cutting lines in the resilient element to form flaps, or cutting a central aperture in the resilient element, or cutting a combination of a central aperture and flaps in the resilient element.

FIG. 10A, FIG. 10B, and FIG. 10C illustrate manufacturing equipment and methods for manufacturing embodiments of the inhaler article 100 having a resilient element 101 affixed to the upstream end 120 of the inhaler article 100. FIG. 10A, FIG. 10B and FIG. 10B illustrate the gluing step of the manufacturing process. FIG. 10A illustrates a step of presenting the resilient element to the glue station 500 and applying the at least one glue ring to the at least one cut disk of resilient material. FIG. 10B illustrates the step of loading the glue ring 502. FIG. 10C illustrates the step of applying glue to the cut disk of resilient material or the resilient element 101. FIG. 10A illustrates the step of presenting the cut disk of resilient material 101 to the glue station 500. The glue stage 501 has a glue ring 502. The resilient element 101 is presented to the glue stage 501 by an element holder 600.

FIG. 10A shows the glue ring 502 before glue has been provided to the glue ring 502. FIG. 10B illustrates the glue stage 501 and the glue ring 502 after glue has been dispensed to the glue ring 502. In other words, the glue ring has been loaded with glue, as shown in FIG. 10B. In embodiments, glue may be dispensed to the glue ring 502 by pressing the glue into the glue ring 502 from a reservoir of glue below the glue stage 501. FIG. 10C illustrates the step of pressing the cut resilient element 101 onto the glue stage 501 to provide glue to glue-side of the resilient element 101. The glue-side of the resilient element is the side that is affixed to the inhaler article 100. FIG. 10C illustrates the step of applying the at least one glue ring to the at least one cut disk of resilient material. Cut disks may be transported to a gluing station by an element holder 600. Glue may be introduced to a gluing stage 500. For example, glue may be applied to the stage. Glue may be pressure-fed to glue rings, sized slightly smaller than the diameter of resilient element disks at the glue stage 500. In embodiments, glue may be presented to the glue ring by pressing the glue into the glue ring from a reservoir of glue below the glue stage. Alternatively, glue may be presented in dots, a discontinuous ring, a thick ring, a thin ring or any other shape to provide glue to the glue station. Appropriate glues may include starch adhesives such as dextrin, casein-based adhesive, polyamide blue, hot melt glue, cyanoacrylate, organic glues, or any other suitable glue.

FIG. 11A, FIG. 11B, and FIG. 11C illustrate manufacturing equipment and methods for manufacturing embodiments of the inhaler article having a resilient element 101 affixed to the upstream end 120 of the inhaler article 100. FIG. 11A illustrates a perspective view of the resilient element 101 having an applied ring of glue 503, after the manufacturing step of FIG. 10C. FIG. 11B illustrates the presentation of the inhaler article 100 to the glue-side of the resilient element 101 having an applied ring of glue 503. FIG. 11B shows the step of presenting the at least one cut disk of resilient material having an applied glue ring 502 to an upstream end 120 of an inhaler article 100 and affixing the at least one cut disk of resilient material 101 having an applied glue ring to the upstream end of an inhaler article to form an inhaler article having an affixed upstream resilient element 101. FIG. 11C illustrates embodiments the inhaler article having a resilient element 101 affixed to the upstream end 120 of the inhaler article 100 at the end of the manufacturing process. FIG. 11C illustrates an embodiment of a resilient element 101 having cuts 104 to form flaps 103, an embodiment of a resilient element 101 having a central aperture 105, and an embodiment of a resilient element 101 having both cuts 104 to form flaps 103 and a central aperture 105. The embodiment of a resilient element 101 having both cuts 104 to form flaps 103 and a central aperture 105 is shown in perspective view and in a top-down view for clarity. In addition, the embodiments of FIG. 11C illustrate the resilient element 101 on an upstream element 18. In embodiments, the upstream element 18 may be present or absent.

For the purpose of the present description and of the appended claims, except where otherwise indicated, all numbers expressing amounts, quantities, percentages, and so forth, are to be understood as being modified in all instances by the term “about”. Also, all ranges include the maximum and minimum points disclosed and include any intermediate ranges therein, which may or may not be specifically enumerated herein. In this context, therefore, a number A is understood as A ±10% of A. Within this context, a number A may be considered to include numerical values that are within general standard error for the measurement of the property that the number A modifies. The number A, in some instances as used in the appended claims, may deviate by the percentages enumerated above provided that the amount by which A deviates does not materially affect the basic and novel characteristic(s) of the claimed invention. Also, all ranges include the maximum and minimum points disclosed and include any intermediate ranges therein, which may or may not be specifically enumerated herein.

1. A method for manufacturing an inhaler article having an upstream resilient element for insertion into a holder to form an inhaler system for providing a dry powder to lungs of a user, the manufacturing method comprising:
moving a ribbon of resilient material from a feeder reel to a cutting stage;
presenting the ribbon of resilient material to the cutting stage;
cutting at least one disk of resilient material from the ribbon of resilient material to form at least one cut disk of resilient material;
presenting the at least one cut disk of resilient material to a glue stage;
dispensing at least one glue ring;
applying the at least one glue ring to the at least one cut disk of resilient material; and,
presenting the at least one cut disk of resilient material having an applied glue ring to an upstream end of an inhaler article;
affixing the at least one cut disk of resilient material having an applied glue ring to the upstream end of an inhaler article to form an inhaler article having an affixed upstream resilient element.

2. The method of claim 1 wherein the cutting step further comprises cutting at least 2 cuts in the at least one disk of resilient material to form at least 4 flaps in each cut disk.

3. The method of claim 1 wherein the cutting step further comprises cutting at least 3 cuts in the at least one disk of resilient material to form at least 6 flaps in each cut disk.

4. The method of claim 1 wherein the cutting step further comprises cutting at least 4 cuts in the at least one disk of resilient material to form at least 8 flaps in each cut disk.

5. The method of claim 2 wherein the cuts extend in a range from about 65% to about 95% of a diameter of the resilient element.

6. The method of claim 1 further comprising cutting a central aperture in the at least one disk of resilient material.

7. The method of claim 6 wherein the central aperture has a diameter which comprises less than 30% of a diameter of the resilient element.

8. The method according to claim 1 further comprising moving the ribbon of resilient material from the cutting stage to a take-up reel.

9. The method of claim 1 further comprising a step of inserting a capsule into the inhaler article prior to the affixing step.

10. The method of claim 1 wherein the resilient element comprises rubber, silicon or latex.

11. The method of claim 1 wherein the inhaler article comprises:
a tubular body extending along a longitudinal axis from a downstream mouthpiece end to an upstream end;
a capsule cavity within the tubular body between the downstream mouthpiece end and the upstream end;
the capsule cavity containing a capsule;
wherein an upstream boundary of the capsule cavity is defined by the resilient element, and
wherein the affixed resilient element retains the capsule in the capsule cavity.

12. The method of claim 11 wherein the downstream mouthpiece end comprises a blocker element.

13. The method of claim 11 wherein the tubular body comprises cardboard.

14. The method of claim 11 wherein the capsule contains pharmaceutically active dry powder comprising nicotine.

15. The method of claim 1 wherein the resilient material comprises color, one or more symbols, or a combination of color and one or more symbols.
